SEQUENCE LISTING (61 sequences)

SEQ ID NO: 1 - protein, 473 aa, Artificial Sequence
  Description of Artificial Sequence Recombinant Protein
MRHKRSAKRTKRASATQLYKTCKQAGTCPPDIIPKVEGKTIAEQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 2 - protein, 200 aa, Artificial Sequence
  Description of Artificial Sequence Recombinant Protein
FDIRGVLDRGPSFKPYSGTAYNSLAPKGAPNTCQWKNANPNVDPNANPNANPNANPSDSKMHTFGAAAMPGVTGKKIEADGLPIRIDSTSGTDTVIYADKTFQPEPQVGNDSWVDTNGAEEKYGGRALKDTTKMKPCYGSFAKPTNKEGGQANLKDSEPSLSTEWSPCSVTCGNGIQVRTIVANYDPDIVMYTENVDLQT

SEQ ID NO: 3 - protein, 204 aa, Artificial Sequence
  Description of Artificial Sequence Recombinant Protein
FDIRGVLDRGPSFKPYSGTAYNSLAPKGAPNTSQWIVTNANPNANPNANPNANPSTKGDNYTFGIASTKGDNITKEGLEIGKDITADNKPIYADKTYQPEPQVGEESWTDIDGTNEKFGGRALKPATKMKPCYGSFARPTNIKGGQAKNRKVTPTEGDVEAEEPDIDMEFFDGREAADAFSPEIVLYTENVNLETPDSHVVYKP

SEQ ID NO: 4 - protein, 200 aa, Artificial Sequence
  Description of Artificial Sequence Recombinant Protein
FDIRGVLDRGPSFKPYSGTAYNSLAPKGAPNTSQWIVTNANPNVDPNANPNANPNANPSTKGDNYTFGIASTKGDNITKEGLEIGKDITADNKPIYADKTYQPEPQVGEESWTDIDGTNEKFGGRALKPATKMKPCYGSFARPTNIKGGQAKNRKVTPTEGDVEAEEPDIDMEFFDGREAADAFSPEIVLYTENVNLETP

SEQ ID NO: 5 - protein, 204 aa, Artificial Sequence
  Description of Artificial Sequence Recombinant Protein
FDIRGVLDRGPSFKPYSGTAYNSLAPKGAPNTSQWIVTTGESTKGDNYTFGIASTKGDNANPNANPNANPNANPADNKPIYADKTYQPEPQVGEESWTDIDGTNEKFGGRALKPATKMKPCYGSFARPTNIKGGQAKNRKVTPTEGDVEAEEPDIDMEFFDGREAADAFSPEIVLYTENVNLETPDSHVVYKPGTSDGNSHANL

SEQ ID NO: 6 - protein, 200 aa, Artificial Sequence
  Description of Artificial Sequence Recombinant Protein
FDIRGVLDRGPSFKPYSGTAYNSLAPKGAPNTSQWIVTTGESTKGDNYTFGIASTKGDNANPNVDPNANPNANPNANPADNKPIYADKTYQPEPQVGEESWTDIDGTNEKFGGRALKPATKMKPCYGSFARPINIKGGQAKNRKVTPTEGDVEAEEPDIDMEFFDGREAADAFSPEIVLYTENVNLETPDSHVVYKPGTS

SEQ ID NO: 7 - protein, 204 aa, Artificial Sequence
  Description of Artificial Sequence Recombinant Protein
FDIRGVLDRGPSFKPYSGTAYNSLAPKGAPNTSQWIVTNANPNANPNANPNANPSTKGDNYTFGIASTKGDNITKEGLEIGKDITADNKPIYADKTYQPEPQVGEESWTDIDGTNEKFGGRALKPATKMKPCYGSFARPTNIKGGQAKNRKVTPSLSTEWSPCSVTCGNGIQVRREAADAFSPEIVLYTENVNLETPDSHVVYK

SEQ ID NO: 8 - protein, 200 aa, Artificial Sequence
  Description of Artificial Sequence Recombinant Protein
FDIRGVLDRGPSFKPYSGTAYNSLAPKGAPNTSQWIVTNANPNVDPNANPNANPNANPSTKGDNYTFGIASTKGDNITKEGLEIGKDITADNKPIYADKTYQPEPQVGEESWTDIDGTNEKFGGRALKPATKMKPCYGSFARPTNIKGGQAKNRKVTPSLSTEWSPCSVTCGNGIQVRREAADAFSPEIVLYTENVNLET

SEQ ID NO: 9 - protein, 204 aa, Artificial Sequence
  Description of Artificial Sequence Recombinant Protein
FDIRGVLDRGPTFKPYSGTAYNALAPKGAPNPCEWDEANANPNANPNANPNANPVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQ

SEQ ID NO: 10 - protein, 200 aa, Artificial Sequence
  Description of Artificial Sequence Recombinant Protein
FDIRGVLDRGPTFKPYSGTAYNALAPKGAPNPCEWDEANANPNVDPNANPNANPNANPVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEG

SEQ ID NO: 11 - protein, 204 aa, Artificial Sequence
  Description of Artificial Sequence Recombinant Protein
FDIRGVLDRGPTFKPYSGTAYNALAPKGAPNPCEWDEAATALEINLEEEDDDNEDEVDEQAEQQKTHVFGQAPYSGINITKEGNANPNANPNANPNANPTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYM

SEQ ID NO: 12 - protein, 200 aa, Artificial Sequence
  Description of Artificial Sequence Recombinant Protein
FDIRGVLDRGPTFKPYSGTAYNALAPKGAPNPCEWDEAATALEINLEEEDDDNEDEVDEQAEQQKTHVFGQAPYSGINITKEGNANPNVDPNANPNANPNANPTFQPEPQIGESQWYETEINHAAGRVLKKTTPMKPCYGSYAKPTNENGGQGILVKQQNGKLESQVEMQFFSTTEATAGNGDNLTPKVVLYSEDVDIET

SEQ ID NO: 13 - protein, 205 aa, Artificial Sequence
  Description of Artificial Sequence Recombinant Protein
FDIRGVLDRGPTFKPYSGTAYNALAPKGAPNPCEWDEANANPNANPNANPNANPVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTESLSTEWSPCSVTCGNGIQVRTPKVVLYSEDVDIETPDTHISYMPTIY

SEQ ID NO: 14 - protein, 200 aa, Artificial Sequence
  Description of Artificial Sequence Recombinant Protein
FDIRGVLDRGPTFKPYSGTAYNALAPKGAPNPCEWDEANANPNVDPNANPNANPNANPVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTESLSTEWSPCSVTCGNGIQVRTPKVVLYSEDVDIETPDT

SEQ ID NO: 15 - protein, 200 aa, Artificial Sequence
  Description of Artificial Sequence Recombinant Protein
FDIRGVLDRGPSFKPYSGTAYNSLAPKGAPNTCQWKNANPNANPNANPSDSKMHTFGAAAMPGVTGKKIEADGLPIRIDSTSGTDTVIYADKTFQPEPQVGNDSWVDTNGAEEKYGGRALKDTTKMKPCYGSFAKPTNKEGGQANLKDSEPAATTPNYDIDLAFFDSKTIVANYDPDIVMYTENVDLQTPDTHIVYKPGT

SEQ ID NO: 16 - protein, 200 aa, Artificial Sequence
  Description of Artificial Sequence Recombinant Protein
FDIRGVLDRGPSFKPYSGTAYNSLAPKGAPNTCQWKNANPNVDPNANPNANPNANPSDSKMHTFGAAAMPGVTGKKIEADGLPIRIDSTSGTDTVIYADKTFQPEPQVGNDSWVDTNGAEEKYGGRALKDTTKMKPCYGSFAKPTNKEGGQANLKDSEPAATTPNYDIDLAFFDSKTIVANYDPDIVMYTENVDLQTPDT

SEQ ID NO: 17 - protein, 200 aa, Artificial Sequence
  Description of Artificial Sequence Recombinant Protein
FDIRGVLDRGPSFKPYSGTAYNSLAPKGAPNTCQWKDSDSKMHTFGAAAMPGVNANPNANPNANPTDTVIYADKTFQPEPQVGNDSWVDTNGAEEKYGGRALKDTTKMKPCYGSFAKPTNKEGGQANLKDSEPAATTPNYDIDLAFFDSKTIVANYDPDIVMYTENVDLQTPDTHIVYKPGTEDTSSESNLGQQAMPNRP

SEQ ID NO: 18 - protein, 200 aa, Artificial Sequence
  Description of Artificial Sequence Recombinant Protein
FDIRGVLDRGPSFKPYSGTAYNSLAPKGAPNTCQWKDSDSKMHTFGAAAMPGVNANPNVDPNANPNANPNANPTDTVIYADKTFQPEPQVGNDSWVDTNGAEEKYGGRALKDTTKMKPCYGSFAKPTNKEGGQANLKDSEPAATTPNYDIDLAFFDSKTIVANYDPDIVMYTENVDLQTPDTHIVYKPGTEDTSSESNLG

SEQ ID NO: 19 - protein, 204 aa, Artificial Sequence
  Description of Artificial Sequence Recombinant Protein
FDIRGVLDRGPSFKPYSGTAYNSLAPKGAPNTCQWKNANPNANPNANPNANPSDSKMHTFGAAAMPGVTGKKIEADGLPIRIDSTSGTDTVIYADKTFQPEPQVGNDSWVDTNGAEEKYGGRALKDTTKMKPCYGSFAKPTNKEGGQANLKDSEPSLSTEWSPCSVTCGNGIQVRTIVANYDPDIVMYTENVDLQTPDTHIVYK

SEQ ID NO: 20 - DNA, 21 bp, Artificial Sequence
  Description of Artificial Sequence Synthetic Oligonucleotide
cggcgtgctggacaggggccc

SEQ ID NO: 21 - DNA, 17 bp, Artificial Sequence
  Description of Artificial Sequence Synthetic Oligonucleotide
gctggctccgtcaaccc

SEQ ID NO: 22 - DNA, 69 bp, Artificial Sequence
  Description of Artificial Sequence Synthetic Oligonucleotide
cattcgggttagcgttaggatttgcgttgggattggcattagcttcatcccattcgcaaggatttgggg

SEQ ID NO: 23 - DNA, 66 bp, Artificial Sequence
  Description of Artificial Sequence Synthetic Oligonucleotide
tcctaacgctaacccgaatgcaaaccccaacgccaatcctgtatttgggcaggcgccttattctgg

SEQ ID NO: 24 - DNA, 70 bp, Artificial Sequence
  Description of Artificial Sequence Synthetic Oligonucleotide
cattcgggttagcgttaggatttgcgttgggattggcattctcagtagttgagaaaaattgcatttccac

SEQ ID NO: 25 - DNA, 59 bp, Artificial Sequence
  Description of Artificial Sequence Synthetic Oligonucleotide
tcctaacgctaacccgaatgcaaaccccaacgccaatcctgcgaccgcaggcaatggtg

SEQ ID NO: 26 - DNA, 74 bp, Artificial Sequence
  Description of Artificial Sequence Synthetic Oligonucleotide
gcattcgggttagcgttaggatttgcgttaggatcgacgttgggattggcattagcttcatcccattcgcaagg

SEQ ID NO: 27 - DNA, 66 bp, Artificial Sequence; identical to SEQ ID NO: 23
  Description of Artificial Sequence Synthetic Oligonucleotide

SEQ ID NO: 28 - DNA, 67 bp, Artificial Sequence
  Description of Artificial Sequence Synthetic Oligonucleotide
ccattcagtgctcagggaattctgaattttattcagatattccgcctcagtagttgagaaaaattgc

SEQ ID NO: 29 - DNA, 61 bp, Artificial Sequence
  Description of Artificial Sequence Synthetic Oligonucleotide
gaattccctgagcactgaatggtcaccttgtagcgtgactttgactcctaaagtggtattg

SEQ ID NO: 30 - DNA, 74 bp, Artificial Sequence; identical to SEQ ID NO: 26
  Description of Artificial Sequence Synthetic Oligonucleotide

SEQ ID NO: 31 - DNA, 62 bp, Artificial Sequence
  Description of Artificial Sequence Synthetic Oligonucleotide
tcctaacgctaacccgaatgcaaaccccaacgccaatcctgctactgctcttgaaataaacc

SEQ ID NO: 32 - DNA, 79 bp, Artificial Sequence
  Description of Artificial Sequence Synthetic Oligonucleotide
cgggtgggcaggtgccggcctgcttgcaggtcttgtacagctgggtggcgctggctctagcttcatcccattcgcaagg

SEQ ID NO: 33 - DNA, 81 bp, Artificial Sequence
  Description of Artificial Sequence Synthetic Oligonucleotide
gcaggccggcacctgcccacccgatatcatccccaaggtggagggcaagaccatcgtatttgggcaggcgccttattctgg

SEQ ID NO: 34 - DNA, 80 bp, Artificial Sequence
  Description of Artificial Sequence Synthetic Oligonucleotide
cgggtgggcaggtgccggcctgcttgcaggtcttgtacagctgggtggcgctggctctctcagtagttgagaaaaattgc

SEQ ID NO: 35 - DNA, 73 bp, Artificial Sequence
  Description of Artificial Sequence Synthetic Oligonucleotide
gcaggccggcacctgcccacccgatatcatccccaaggtggagggcaagaccatcgcgaccgcaggcaatggt

SEQ ID NO: 36 - DNA, 80 bp, Artificial Sequence; identical to SEQ ID NO: 34
  Description of Artificial Sequence Synthetic Oligonucleotide

SEQ ID NO: 37 - DNA, 76 bp, Artificial Sequence
  Description of Artificial Sequence Synthetic Oligonucleotide
gcaggccggcacctgcccacccgatatcatccccaaggtggagggcaagaccatcactcctaaagtggtattgtac

SEQ ID NO: 38 - protein, 941 aa, Artificial Sequence
  Description of Artificial Sequence Recombinant Protein
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEANANPNANPNANPNANPNANPGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 39 - protein, 972 aa, Artificial Sequence
  Description of Artificial Sequence Recombinant Protein
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTENANPNANPNANPNANPNANPATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 40 - protein, 947 aa, Artificial Sequence
  Description of Artificial Sequence Recombinant Protein
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEANANPNVDPNANPNANPNANPNANPVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 41 - protein, 965 aa, Artificial Sequence
  Description of Artificial Sequence Recombinant Protein
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAEYLNKIQNSLSTEWSPCSVTLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 42 - protein, 976 aa, Artificial Sequence
  Description of Artificial Sequence Recombinant Protein
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEANANPNVDPNANPNANPNANPNANPATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 43 - protein, 953 aa, Artificial Sequence
  Description of Artificial Sequence Recombinant Protein
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEARASATQLYKTCKQAGTCPPDIIPKVEGKTIVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 44 - protein, 982 aa, Artificial Sequence
  Description of Artificial Sequence Recombinant Protein
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTERASATQLYKTCKQAGTCPPDIIPKVEGKTIATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 45 - protein, 973 aa, Artificial Sequence
  Description of Artificial Sequence Recombinant Protein
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTERASATQLYKTCKQAGTCPPDIIPKVEGKTITPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 46 - protein, 76 aa, Artificial Sequence
  Description of Artificial Sequence Synthetic peptide
NANPNANPNANPNANPNANPNANPNANPNANPNANPNANPNANPNANPNANPNANPNANPNANPNANPNANPNANP

SEQ ID NO: 47 - protein, 36 aa, Artificial Sequence
  Description of Artificial Sequence Synthetic peptide
NANPNVDPNANPNANPNVDPNANPNANPNVDPNANP

SEQ ID NO: 48 - protein, 16 aa, Artificial Sequence
  Description of Artificial Sequence Synthetic peptide
NANPNANPNANPNANP

SEQ ID NO: 49 - protein, 20 aa, Artificial Sequence
  Description of Artificial Sequence Synthetic peptide
NANPNVDPNANPNANPNANP

SEQ ID NO: 50 - protein, 20 aa, Artificial Sequence
  Description of Artificial Sequence Synthetic peptide
SLSTEWSPCSVTCGNGIQVR

SEQ ID NO: 51 - protein, 40 aa, Artificial Sequence
  Description of Artificial Sequence Synthetic peptide
NANPNANPNANPNANPNANPNANPNANPNANPNANPNANP

SEQ ID NO: 52 - protein, 96 aa, Artificial Sequence
  Description of Artificial Sequence Synthetic peptide
NANPNVDPNANPNANPNVDPNANPNANPNVDPNANPNANPNVDPNANPNANPNVDPNANPNANPNVDPNANPNANPNVDPNANPNANPNVDPNANP

SEQ ID NO: 53 - protein, 20 aa, Artificial Sequence
  Description of Artificial Sequence Synthetic peptide
EYLNKIQNSLSTEWSPCSVT

SEQ ID NO: 54 - protein, 45 aa, Artificial Sequence
  Description of Artificial Sequence Synthetic peptide
GDRAAGQPAGDRAAGQPAGDRAAGQPAGDRAAGQPAGDRAAGQPA

SEQ ID NO: 55 - protein, 45 aa, Artificial Sequence
  Description of Artificial Sequence Synthetic peptide
ANGAGNQPGANGAGNQPGANGAGNQPGANGAGNQPGANGAGNQPG

SEQ ID NO: 56 - protein, 44 aa, Artificial Sequence
  Description of Artificial Sequence Synthetic peptide
APGANQEGGAAAPGANQEGGAAAPGANQEGGAAAPGANQEGGAA

SEQ ID NO: 57 - protein, 20 aa, Artificial Sequence
  Description of Artificial Sequence Synthetic peptide
NANPNANPNANPNANPNANP

SEQ ID NO: 58 - protein, 48 aa, Artificial Sequence
  Description of Artificial Sequence Synthetic peptide
NANPNVDPNANPNANPNVDPNANPNANPNVDPNANPNANPNVDPNANP

SEQ ID NO: 59 - protein, 30 aa, Artificial Sequence
  Description of Artificial Sequence Synthetic peptide
RASATQLYKTCKQAGTCPPDIIPKVEGKTI

SEQ ID NO: 60 - protein, 4 aa, Artificial Sequence
  Description of Artificial Sequence Synthetic peptide
NANP

SEQ ID NO: 61 - protein, 4 aa, Artificial Sequence
  Description of Artificial Sequence Synthetic peptide
NVDP

(19) United States
(12) Patent Application Publication
(10) Pub. No.: US 2010/0272753 A1
(43) Pub. Date: Oct. 28, 2010

(54) Recombinant Adenovirus Vaccines

(75) Inventors: Gary W. Ketner, Baltimore, MD (US); Richard B. Roden, Baltimore, MD (US); Fidel P. Zavala, Baltimore, MD (US)

Correspondence Address: VENABLE LLP, P.O. BOX 34385, WASHINGTON, DC, 20043-9998, US

(73) Assignee: The Johns Hopkins University, Baltimore, MD (US)

(21) Appl. No.: 12/447,357

(22) PCT Filed: Oct. 26, 2007
(86) PCT No.: PCT/US07/22745

§ 371 Date: Jun. 17, 2010

Related U.S. Application Data
(60) Provisional application No. 60/854,876, filed on Oct. 26, 2006.

(51) Int. Cl. A61K 39/235; A61K 35/76; C12N 7/01

(52) U.S. Cl. 424/233.1; 424/93.2; 435/235.1

(57) ABSTRACT

Recombinant adenovirus vaccines comprising recombinant adenoviruses whose hexon, fiber or protein IX capsid proteins are engineered to include exogenous peptide segments, e.g. vaccines for human papillomavirus (HPV) and malaria.

The invention disclosed herein was made in part with funds from the U.S. Government, Grant Nos. P50 CA098252, AI025239, GM082127, CA098252. The U.S. Government has certain rights in the invention.

### BACKGROUND

1. Field of the Invention

The invention relates to recombinant adenovirus vaccines comprising recombinant adenoviruses whose hexon, fiber or protein IX capsid proteins are engineered to include exogenous peptide segments, e.g. protective epitopes for human papillomavirus (HPV) and malaria.

2. Background Information

Despite many decades of research advances, infectious disease remains a major public health problem, exacting a severe toll on both individuals and society. Acute and chronic infection impacts millions of people world wide each year, having both immediate and long term consequences. Vaccines have shown promise, but in many cases have failed to provide full protection against the target organism(s).

Cervical cancer caused by HPV infection kills about 200,000 women annually. The currently licensed HPV vaccine, GARDASIL®, although effective, protects against only a subset of the multiple HPV types that induce disease. Furthermore, existing papillomavirus vaccines are relatively expensive to produce and administer and require repeat injections.

Malaria is a world-wide major public health problem, with approximately 200 million cases of malaria reported yearly, and 3 million deaths. Efforts to develop effective controls against the mosquito vector using aggressive applications of pesticides ultimately led to the development of pesticide resistance. Similarly, efforts at treatment of the disease through anti-parasitic drugs led to parasite drug-resistance. As the anti-vector and anti-parasite approaches failed, efforts have become focused on malaria vaccine development as an effective and inexpensive alternative approach.

Leading malaria circumsporozoite (CSP) peptide-based malaria vaccine candidates consist of purified virus-like particles (VLPs) formed from either recombinant hepatitis B core or recombinant hepatitis B surface antigens engineered to contain the malaria peptides. Two VLP-based candidate vaccines that incorporate CSP peptide antigens (RTS,S and ICC-1132) have shown partial efficacy in human clinical trials. These vaccines must be injected and do not replicate in the vaccinated individual. Furthermore they require multiple doses, typically with adjuvants, and must be highly purified from recombinant E. coli or yeast expression systems.

Thus, there is a need for new types of vaccines that have improved efficacy and ease of administration.

### BRIEF DESCRIPTION OF THE DRAWINGS

FIG. 1. Hexon modification by overlap PCR. (A) Hexon DNA is used as template in two separate PCR reactions. The primer pair for one reaction is indicated above the line; the primer pair for the other below. One member of each primer pair is complementary to hexon DNA (upstream outside or downstream outside primers). The other contains sequences complementary to the hexon DNA immediately adjacent to the site of insertion/substitution and sequences encoding overlapping portions of the desired substitution/insertion sequences (5′ mutagenic or 3′ mutagenic primers). These PCR reactions yield DNA fragments each containing hexon sequences and a portion of the substitution/insertion, overlapping in the substitution/insertion region (B). A second round of PCR using the original outside primers and a mixture of overlapping fragments as template generates a DNA fragment that extends between the outside primers and contains the desired substitution/insertion. Creation of a substitution is shown in the figure. Blue lines indicate adenovirus sequences, red lines substitution sequences.

FIG. 2. Inserted epitopes are present in hexon and on adenovirus particles. Top left. Immunoblots with Ad5 late protein antiserum (α-Ad5 late) and anti-NANP monoclonal antibody (α-NANP MAb) of Ad5 and NANP/NVDP (SEQ ID NOS 60-61) capsid display recombinant proteins. Lanes contain either purified virions (Vir.) or infected cell lysates (lys.). The positions of major adenovirus capsid proteins are marked on the left (IIIA and fiber co-migrate) and the positions of II-g and G2 hexon proteins on the right. G2 hexon is a net 14 amino acid (14aa) deletion and the II-g hexon is a net 24aa insertion, accounting for the difference in mobility of the two recombinant hexon proteins. The three panels are from different blots and are not vertically aligned. Top right. Immunoblot of Ad5 and HPV L2 capsid display recombinant virion proteins with HPV L2 17-36 monoclonal antibody RG-1 (above) and anti-Ad5 late protein serum (below). Only the hexon region of the gel is shown. Bottom. Immunogold labeling of NANP capsid display recombinant G2. Purified G2 or wild type Ad5 virus particles were reacted first with anti-NANP monoclonal antibody and then with secondary antibody conjugated to 2 nm gold beads (arrows). Negatively-stained electron micrographs show that the recombinant (A) but not the Ad5 virions (B) are reactive with the NANP MAb.

FIG. 3. NANP Capsid display antisera recognize authentic CSP. Whole sporozoite lysates were immunoblotted with pre-immune mouse serum (p.i.) or serum from mice immunized with Ad5 or NANP capsid display recombinant G2. The lane marked ‘2A10’ was blotted with an NANP-specific monoclonal. Arrow: position of CSP.

FIG. 4. NANP capsid display antisera recognize sporozoites. P. falciparum sporozoites were reacted with antiserum from mice immunized with the NANP capsid display recombinant G2 (left) or with Ad5 (right). Slides were stained with FITC-conjugated secondary antibody and examined by fluorescent microscopy. G2 antiserum stains sporozoites at dilutions of 1:1000-1:8000 (1:2000 shown); Ad5 serum is not reactive at 1:1000.

FIG. 5. Sporozoite neutralization by NANP capsid display immunization. Mice were immunized with NANP capsid display recombinant G2. Immune (G2) and control sera were incubated in vitro with transgenic P. berghei sporozoites carrying the P. falciparum CSP NANP repeat and the mixtures were added to liver cells in culture. Parasite replication was measured 72 h post-infection by qPCR quantitation of P. berghei 18S rRNA in infected cells. Replication is expressed as the ratio between parasite rRNA and human actin in infected cells. Reduced ratios indicate that neutralization occurred. Controls included pre-immune serum, NANP-specific monoclonal antibody (MAb), and serum from mice immunized with Ad5. The right-most bar shows the 18S rRNA present in cells infected with killed (gamma-irradiated) sporozoites. Ratios are the average of two biological replicates, each determined by three technical replicates. Error bars are the standard deviation of the mean of the two biological replicates.

FIG. 6. HPV16 L2 17-36 peptide ELISA of mouse sera at 21 days (one week after second immunization). Immobilon plates (Nunc) were coated with 100 ng/well of HPV16 L2 17-36 peptide in PBS overnight at 4° C. Wells were then blocked with 1% bovine serum albumin (BSA)-PBS for 1 h at room temperature, and incubated with 2-fold dilutions of mouse sera for 1 h at room temperature. Following a wash step with PBS-0.01% (v/v) Tween 20, peroxidase-labeled goat anti-mouse IgG (KPL Inc, Gaithersburg, Md.) diluted 1:5,000 in 1% BSA-PBS was added for 1 h. The plates were then washed and developed with 2,2′-azino-bis(3-ethylbenzthiazoline-6-sulphonic acid solution (Roche) for 10 min. Titers <50 were not considered significant.

FIG. 7. In vitro HPV16 neutralization titers for sera collected at day 42 (two weeks after third immunization). The HPV16 pseudovirion in vitro neutralization assay was performed as described earlier in Pastrana et al, and the secreted alkaline phosphatase content in the clarified supernatant was determined using the p-Nitrophenyl phosphate tablets (Sigma, St. Louis, Mo.) dissolved in diethanolamine and absorbance measured at 405 nm. Constructs and detailed protocols for the preparation of the pseudovirions can be found at http://home.ccr.cancer.gov/lco/. Titers were defined as the reciprocal of the highest dilution that caused a 50% reduction in A₄₀₅, and a titer <50 was not considered significant. Titers >102400 are listed as 204800.

FIG. 8. HPV16 cutaneous challenge study. Mice were challenged on their belly with HPV16 pseudovirions carrying the luciferase reporter gene at day 44 (16 days after the third immunization). Three days later the mice were injected with luciferin, imaged (left panel) and bioluminescence quantified in relative light units (right panel). HPV16 pseudovirus was prepared as described in Gambhira et al, 2007 in press by packaging a luciferase expression construct (see http://home.ccr.cancer.gov/lco/ for plasmid maps and production methods). A patch on the belly of anesthetized Balb/c mice was shaved with an electric razor without traumatizing the epithelium. Challenge was performed by application to the shaved skin of 3×10⁹ HPV16 pseudovirions (100 ng) in 10 μl 0.6% carboxymethylcellulose (Sigma C5013) containing L1 and L2 (or L1 alone for background determination) and carrying an encapsidated luciferase reporter construct. Three days later, the mice were anesthetized, injected with luciferin (100 μl at 7 mg/ml) and their image acquired for 10 min with a Xenogen IVIS 200.

FIG. 9. Quantification of HPV16 cutaneous challenge study. Equal areas encompassing the site of inoculation were analyzed using Living Image 2.20 software, and background was determined by challenge with non-infectious HPV pseudovirions lacking L2. Bioluminescence was qualified in relative light units (RLU).

### DESCRIPTION OF THE INVENTION

Described herein are recombinant adenoviruses whose hexon, fiber or protein IX capsid proteins are engineered to include exogenous peptide segments. The recombinant adenoviruses are useful in formulating “capsid-display vaccines”, wherein the exogenous peptide segments are displayed on the exterior of the adenovirus particles, and induce immunity to, e.g., microorganisms from which the exogenous peptide segments are derived. In one aspect, the recombinant adenoviruses described herein are viable, replicate in individuals to whom they are administered, e.g. as vaccines, and induce immunity.

In one general embodiment, a recombinant adenovirus is provided whose hexon, fiber or protein IX capsid proteins are engineered to include peptide segments derived from a papillomavirus minor capsid protein (L2). The L2 segment may be obtained from any non-human animal papillomavirus, e.g. bovine papillomavirus type 1 (BPV1), or a human papillomavirus, for example, L2 from HPV16, set forth as follows:

| (SEQ ID NO: 1) | |
|---|---|
| 1 | mrhkrsakrt krasatqlyk tckqagtcpp diipkvegkt iaeqilqygs mgvffgglgi |
| 61 | gtgsgtggrt gyiplgtrpp tatdtlapvr ppltvdpvgp sdpsivslve etsfidagap |
| 121 | tsvpsippdv sgfsittstd ttpaildinn tvttvtthnn ptftdpsvlq pptpaetggh |
| 181 | ftlssstist hnyeeipmdt fivstnpntv tsstpipgsr pvarlglysr ttqqvkvvdp |
| 241 | afvttptkli tydnpayegi dvdntlyfss ndnsiniapd pdfldivalh rpaltsrrtg |
| 301 | irysrignkq tlrtrsgksi gakvhyyydl stidpaeeie lqtitpstyt ttshaaspts |
| 361 | innglydiya ddfitdtstt pvpsvpstsl sgyipantti pfggaynipl vsgpdipini |
| 421 | tdqapslipi vpgspqytii adagdfylhp syymlrkrrk rlpyffsdvs laa |

In another embodiment, the L2 sequence is a consensus sequence of two or more different papillomavirus types, for example a sequence with 95%, or 90% or 80% amino acid homology to L2 of any papillomavirus type. In yet another embodiment, multiple neutralizing epitopes from within L2 are linked together (i.e. by eliminating intervening non-neutralizing epitopes) with or without spacers between each epitope, in any order and from any papillomavirus type.
It has been found that the L2 segment induces a multitypic immunity, protecting against most or all HPV types. In addition, live vaccines using this design should have advantages of low cost of production and administration, and are expected to confer protection with a single oral dose.

Accordingly, it is one object to provide a recombinant adenovirus comprising a polynucleotide encoding a papillomavirus L2 peptide segment of human or bovine (other animal papillomavirus type as there are possible veterinary uses) origin, preferably inserted into or replacing at least one portion of a DNA sequence encoding an adenovirus surface-exposed protein.

By “portion” of a DNA sequence is meant a part of the sequence that is at least 3 bases up to about 150 nucleotide bases in length. In some cases, two or more portions of DNA sequences encoding an adenovirus surface protein may have such insertions or replacements.

L2 segments to be inserted or substituted into the capsid proteins may be of any length, but are usually at least about 5 amino acid residues up to about 40 residues. Larger segments, e.g. 50, 60, 70, or 80 residues, up to and including the full length L2 may be useful. (Gambhira et al. J. Virol., November 2007) (Unless otherwise stated or clearly inapplicable, stated ranges herein are intended to include all integer values within the range, e.g. “1-5” includes 1, 2, 3, 4, and 5.)

In specific embodiments, the HPV L2 peptide segment comprises L2 amino acid numbers 17-36, 64-81 and/or 94-122.

Also provided is a recombinant adenovirus wherein the L2 peptide segment is flanked by spacer peptide(s). A spacer peptide may be joined to the N terminus and/or the C terminus of the L2 peptide segment, and may consist of a peptide tag, e.g. from the group including, but not limited to, FLAG, myc, Poly-Arginine, Poly-Histidine, Strep-tag II, Maltose-binding domain, VSV-G, V5, HSV, influenza HA, and Glutathione-S-transferase.

The recombinant adenovirus may be of any suitable type, as will be apparent to those of skill in the art, including, but not limited to:
a) human adenovirus type 2;
b) human adenovirus type 4;
c) human adenovirus type 5;
d) human adenovirus type 7;
e) human adenovirus type 21;
f) human adenovirus type 35;
g) chimpanzee adenovirus type AdC7; and
h) chimpanzee adenovirus type AdC68.

The papillomavirus L2 peptide segment may be derived from, for example:
a) Human papillomavirus-16;
b) Human papillomavirus-18;
c) Human papillomavirus-6;
d) a member of the genus Alpha-papillomavirus;
e) a member of the genus Beta-papillomavirus; and
f) Bovine papillomavirus type 1.

In one particular embodiment, the L2 segment is derived from Human Papillomavirus-16.

The L2 peptide segment may be inserted, for example, into one of hexon hypervariable regions 1-7, fiber HI loop, or the peptide segment may be attached, with an optional linker, to the carboxy terminus of protein IX capsid proteins.

For example, amino acid residues 17-36 of HPV L2 may be inserted into human adenovirus type 2 hexon hypervariable region 1 amino acids 139-174, human adenovirus type 4 hexon hypervariable region 1 amino acids 139-143, human adenovirus type 5 hexon hypervariable region 1 amino acids 139-167, human adenovirus type 7 hexon hypervariable region 1 amino acids 139-147, human adenovirus type 21 hexon hypervariable region 1 amino acids 139-158, human adenovirus type 35 hexon hypervariable region 1 amino acids 139-162, chimpanzee adenovirus type AdC7 hexon hypervariable region 1 amino acids 134-143, chimpanzee adenovirus type AdC68 hexon hypervariable region 1 amino acids 139-149, human adenovirus type 2 hexon hypervariable region 2 amino acids 191-209, human adenovirus type 4 hexon hypervariable region 2 amino acids 163-177, human adenovirus type 5 hexon hypervariable region 2 amino acids 184-198, human adenovirus type 7 hexon hypervariable region 2 amino acids 164-181, human adenovirus type 21 hexon hypervariable region 2 amino acids 178-196, human adenovirus type 35 hexon hypervariable region 2 amino acids 180-199, chimpanzee adenovirus type AdC7 hexon hypervariable region 2 amino acids 160-175, chimpanzee adenovirus type AdC68 hexon hypervariable region 2 amino acids 166-181, human adenovirus type 2 hexon hypervariable region 5 amino acids 283-292, human adenovirus type 4 hexon hypervariable region 5 amino acids 229-263, human adenovirus type 5 hexon hypervariable region 5 amino acids 272-280, human adenovirus type 7 hexon hypervariable region 5 amino acids 262-266, human adenovirus type 21 hexon hypervariable region 5 amino acids 275-279, human adenovirus type 35 hexon hypervariable region 5 amino acids 277-281, chimpanzee adenovirus type AdC7 hexon hypervariable region 5 amino acids 251-256, chimpanzee adenovirus type AdC68 hexon hypervariable region 5 amino acids 257-262, human adenovirus type 2 fiber HI loop amino acids 537-550, human adenovirus type 4 fiber HI loop amino acids 385-393, human adenovirus type 5 fiber HI loop amino acids 537-549, human adenovirus type 7 fiber HI loop amino acids 278-287, human adenovirus type 21 fiber HI loop amino acids 277-286, human adenovirus type 35 fiber HI loop amino acids 277-286, chimpanzee adenovirus type AdC7 fiber HI loop amino acids 403-411, or chimpanzee adenovirus type AdC68 fiber HI loop amino acids 385-393.

Thus, in specific embodiments, the L2 peptide segment is selected from the group consisting of:
a) Full-length L2;
b) Amino acids 17-36;
c) Amino acids 65-81;
d) Amino acids 94-122
e) Amino acids 1-88; and
f) Amino acids 11-200.

The peptide segment may be attached, with an optional linker, e.g. to the human adenovirus type 2 protein IX amino acid 140, the human adenovirus type 4 protein IX amino acid 142, the human adenovirus type 5 protein IX amino acid 140, the human adenovirus type 7 protein IX amino acid 138, the human adenovirus type 21 protein IX amino acid 139, the human adenovirus type 35 protein IX amino acid 139, the chimpanzee adenovirus type ADC7 protein IX amino acid 142, the chimpanzee adenovirus type ADC68 protein IX amino acid 142.

The L2 peptide segment may be either inserted into or replace at least a portion of an adenoviral surface protein selected from the group consisting of:
a) hexon;
b) fiber; and
c) protein IX capsid proteins.
   Where replacement occurs, the inserted L2 peptide segment may be equal to, larger or smaller than the portion of the adenoviral surface protein that is replaced.

In specific embodiments, the L2 peptide segment replaces at least a portion of hexon hypervariable region 1, least a portion of hexon hypervariable region 2, at least a portion of hexon hypervariable region 5, or at least a portion of the fiber HI loop.

For example, amino acids 17-36 of HPV L2 may replace at least a portion of human adenovirus type 2 hexon hypervariable region 1 amino acids 139-174, amino acids 17-36 of HPV L2 may replace at least a portion of human adenovirus type 4 hexon hypervariable region 1 amino acids 139-143, amino acids 17-36 of HPV L2 may replace at least a portion of human adenovirus type 5 hexon hypervariable region 1 amino acids 139-167, amino acids 17-36 of HPV L2 may replace at least a portion of human adenovirus type 7 hexon hypervariable region 1 amino acids 139-147, amino acids 17-36 of HPV L2 may replace at least a portion of human adenovirus type 21 hexon hypervariable region 1 amino acids 139-158, amino acids 17-36 of HPV L2 may replace at least a portion of human adenovirus type 35 hexon hypervariable region 1 amino acids 139-162, amino acids 17-36 of HPV L2 may replace at least a portion of chimpanzee adenovirus type AdC7 hexon hypervariable region 1 amino acids 134-143, amino acids 17-36 of HPV L2 may replace at least a portion of chimpanzee adenovirus type AdC68 hexon hypervariable region 1 amino acids 139-149, amino acids 17-36 of HPV L2 may replace at least a portion of human adenovirus type 2 hexon hypervariable region 2 amino acids 191-209, amino acids 17-36 of HPV L2 may replace at least a portion of human adenovirus type 4 hexon hypervariable region 2 amino acids 163-177, amino acids 17-36 of HPV L2 may replace at least a portion of human adenovirus type 5 hexon hypervariable region 2 amino acids 184-198, amino acids 17-36 of HPV L2 may replace at least a portion of human adenovirus type 7 hexon hypervariable region 2 amino acids 164-181, amino acids 17-36 of HPV L2 may replace at least a portion of human adenovirus type 21 hexon hypervariable region 2 amino acids 178-196, amino acids 17-36 of HPV L2 may replace at least a portion of human adenovirus type 35 hexon hypervariable region 2 amino acids 180-199, amino acids 17-36 of HPV L2 may replace at least a portion of chimpanzee adenovirus type AdC7 hexon hypervariable region 2 amino acids 160-175, amino acids 17-36 of HPV L2 may replace at least a portion of chimpanzee adenovirus type AdC68 hexon hypervariable region 2 amino acids 166-181, amino acids 17-36 of HPV L2 may replace at least a portion of human adenovirus type 2 hexon hypervariable region 5 amino acids 283-292, amino acids 17-36 of HPV L2 may replace at least a portion of human adenovirus type 4 hexon hypervariable region 5 amino acids 229-263, amino acids 17-36 of HPV L2 may replace at least a portion of human adenovirus type 5 hexon hypervariable region 5 amino acids 272-280, amino acids 17-36 of HPV L2 may replace at least a portion of human adenovirus type 7 hexon hypervariable region 5 amino acids 262-266, amino acids 17-36 of HPV L2 may replace at least a portion of human adenovirus type 21 hexon hypervariable region 5 amino acids 275-279, amino acids 17-36 of HPV L2 may replace at least a portion of human adenovirus type 35 hexon hypervariable region 5 amino acids 277-281, amino acids 17-36 of HPV L2 may replace at least a portion of chimpanzee adenovirus type AdC7 hexon hypervariable region 5 amino acids 251-256, amino acids 17-36 of HPV L2 may replace at least a portion of chimpanzee adenovirus type AdC68 hexon hypervariable region 5 amino acids 257-262, amino acids 17-36 of HPV L2 may replace at least a portion of human adenovirus type 2 fiber HI loop amino acids 537-550, amino acids 17-36 of HPV L2 may replace at least a portion of human adenovirus type 4 fiber HI loop amino acids 385-393, amino acids 17-36 of HPV L2 may replace at least a portion of human adenovirus type 5 fiber HI loop amino acids 537-549, amino acids 17-36 of HPV L2 may replace at least a portion of human adenovirus type 7 fiber HI loop amino acids 278-287, amino acids 17-36 of HPV L2 may replace at least a portion of human adenovirus type 21 fiber HI loop amino acids 277-286, amino acids 17-36 of HPV L2 may replace at least a portion of human adenovirus type 35 fiber HI loop amino acids 277-286, amino acids 17-36 of HPV L2 may replace at least a portion of chimpanzee adenovirus type AdC7 fiber HI loop amino acids 403-411, amino acids 17-36 of HPV L2 may replace at least a portion of chimpanzee adenovirus type AdC68 fiber HI loop amino acids 385-393.

The recombinant adenoviruses provided herein are in general capable of replicating in cells, in particular in a mammalian host, for example, a human, and of inducing an immune response. In some instances, however, defective or attenuated recombinant adenoviruses may be constructed, which are incapable of replication. This can be accomplished by means known to those of skill in the art, for example, through chemical inactivation (e.g. using UV or psoralen, or other chemical cross-linker), as well as genetic inactivation by deletion or selective mutation of functions critical for replication, and complementing the mutation for manufacture of the construct. These modifications may increase the safety of the construct in immunocompromised hosts. A non-human animal adenovirus also may be used. Alternatively, defective or attenuated adenoviruses might be used if the construct was intended to be injected, and/or expressed therapeutic antigens (e.g. any HPV early antigen).

In one specific embodiment, the immune response is directed to the HPV L2 segment. The immune response may be mediated e.g. by antibody or T cells, and will preferably prevent infection with HPV. In a preferred embodiment, the immune response provides sterilizing immunity to HPV.

Also provided are compositions and vaccines comprising the recombinant adenovirus disclosed herein, and methods of vaccination against HPV or malaria using the compositions.

Thus, in specific embodiments, a pharmaceutical composition and/or vaccine is provided comprising a recombinant adenovirus as described herein, and a method of vaccination against Human papillomavirus comprising administering a composition comprising the recombinant adenovirus such that an immune response occurs in the subject. Administration may be by any suitable route, for example, intramuscular, intradermal, subcutaneous, intra-nasal, vaginal, anal, oral, etc. In a preferred embodiment, administration is oral.

It will be appreciated that a pharmaceutical composition or vaccine comprising the recombinant adenovirus may contain adjuvants, excipients and carriers, and use modes of delivery that are customary to facilitate administration and improve efficacy. In one preferred embodiment, enteric coated capsules or tablets are formulated for oral administration. Further detail may be found, e.g. in Remington's Pharmaceutical Sciences,” 1990, 18th ed., Mack Publishing Co., Easton, Pa.

It will also be appreciated that the recombinant adenoviruses can be designed and made to include multiple insertions of L2 and/or malarial peptide segments, as described herein, as well as other nonadenoviral peptide segments, peptides, polypeptides or proteins, e.g. for the purpose of obtaining constructs conferring more broad based immunity and/or producing multivalent vaccines.

The terms “peptide”, “polypeptide”, and “protein” are used interchangeably herein, unless context clearly indicates otherwise. A “peptide segment” refers to a portion of a defined peptide (e.g. L2 or CSP).

In another general embodiment, a recombinant adenovirus is provided whose hexon, fiber or protein IX capsid proteins are engineered to include peptide segments from a malaria protein, for example, a malaria circumsporozoite protein.

The malaria vaccine described herein differs from existing adenovirus-based recombinant malaria vaccines in expressing specific CSP peptides on adenovirus particles produced by replication in the vaccinee. Other adenovirus-based malaria vaccine candidates express malaria antigens (CSP or others) intracellularly. Additionally, other adenovirus-based malaria vaccine candidates are defective and do not replicate in vaccinees, requiring immunization by injection; probably in multiple doses.

Thus, the vaccine differs from existing malaria vaccines that employ the same or similar antigenic peptides in being in an adenovirus background, being replication-competent in vaccinees, and being capable of oral administration. Replication of the viable adenovirus vaccines in the vaccinee potentially increases effectiveness, induces a broader spectrum of immune responses, and reduces costs by eliminating the need for multiple doses, syringes, and highly trained personnel.

Display of malaria antigen peptides on adenovirus particles, as detailed herein, could be combined with other expression technologies to enhance or broaden the immune response of adenovirus-based malaria vaccines. For example, used in concert with MLTU-based (major late transcriptional unit-based) live recombinants expressing the malaria LSA-3 antigen, capsid display of CSP peptides would target two pre-erythrocytic malaria antigens known to be protective in animal systems (Berg et al. PNAS, 2005; Berg et al, Vaccine, 2007). Alternatively, CSP capsid-display in concert with MLTU-based expression of a blood-stage antigen could target both the pre-erythrocytic and erythrocytic stages of malaria infection. The capsid-display strategy could also be combined with defective adenovirus-based malaria vaccination strategies with similar beneficial effects.

Of the antigens currently under consideration for use in malaria vaccines, only the circumsporozoite protein (CSP) has been successful in inducing protection from malaria infection in humans. Two VLP-based candidate vaccines that incorporate CSP peptide antigens (RTS,S and ICC-1132) have shown partial efficacy in human clinical trials. The RTS,S and ICC-1132 candidate vaccines, although composed of different viral proteins, bear similar CSP antigens: a repeating peptide related to the R-region NANP repeat ([NANP]₁₉ (SEQ ID NO:46) for RTS,S and NANPNVDP[NANP]₃ (SEQ ID NO:47) for ICC-1132), and an amino acid segment derived from the carboxyl terminus of CSP (amino acids 207-395, RTS,S; 326-345, ICC-1132). Three or more NANP repeats constitute a B-cell epitope that induces neutralizing antibody in rabbits, NANPNVDP[NANP]₃ (SEQ ID NO:47) contains both B- and T-cell epitopes, and the carboxyterminal region of CSP contains a ‘universal’ T-cell epitope (T*) that binds to a broad range of MHC Class II molecules (Zavala, Tam et al. 1985; Nardin, Herrington et al. 1989; Moreno, Clavijo et al. 1993; Nardin, Calvo-Calle et al. 2001; Walther, Dunachie et al. 2005). Therefore, together, these peptides induce both humoral and cell-mediated responses to CSP. Because of the demonstrated success of VLP vaccines containing these peptides, the recombinant adenovirus vaccines described here can also employ NANP-related and T* epitopes. To avoid potential problems with the insertion of very long peptides into hexon, the shorter peptides present in ICC-1132 can be used to prepare capsid-display recombinants. Recombinants can bear (NANP)₄ alone, the NANPNVDP(NANP)₃ (SEQ ID NO:47) B/T-cell epitope alone, and a combination of the NANPNVDP(NANP)₃ (SEQ ID NO:47) and T* epitopes. The CSP peptides can be inserted into hypervariable regions (HVRs) 1, 2 and 5 in the hexon protein (Rux, Kuser et al. 2003). HVR5 has been shown to be capable of accommodating an 14 as peptide (Worgall, Krause et al. 2005), similar in size to the 12 to 20 amino acid peptides described here. For HVR1 and 2 detailed comparative analysis of adenovirus hexons (Rum, Kuser et al. 2003) suggests that they can accommodate peptides of the proposed length. In the event that recombinants cannot be recovered using these HVRs, additional sites that can accommodate insertions have been predicted and can be tested. Construction of modified hexon genes can be done by PCR-based modification of cloned segments of the gene. Modified segments then can be incorporated into intact viral DNA by ligation to purified genomic terminal fragments. Exemplary hexon protein sequences, incorporating the inserted malaria CSP sequences are presented below

It is envisioned that the adenovirus-based vaccines described herein will be prepared by modification of the adenovirus type 4 and/or type 7 vaccine strains, will be formulated in enteric-coated capsules, and will be administered by a single oral dose.

Typical modified adenovirus hexon protein sequences proposed for capsid-display malaria vaccines. Serotype, CSP peptide, and insertion location is noted for each sequence. Ad5: adenovirus type 5, Ad4: Adenovirus type 4, Ad7: adenovirus type 7; NANP: NANPNANPNANPNANP (SEQ ID NO:48); NVDP: NANPNVDPNANPNANPNANP (SEQ ID NO:48), T*: SLSTEWSPCSVTCGNGIQVR (SEQ ID NO:50); HVR: hypervariable region. Malaria peptides are underlined. Amino acids 101-300 (out of about 950) are shown for each modified hexon protein. The remainder of the protein is identical to wild-type hexon.

Ad4 NVDP HVR1, T* HVR5

(SEQ ID NO: 2)

FDIRGVLDRGPSFKPYSGTAYNSLAPKGAPNTCQWKNANPNVDPNANPNA
NPNANPSDSKMHTFGAAAMPGVTGKKIEADGLPIRIDSTSGTDTVIYADK
TFQPEPQVGNDSWVDTNGAEEKYGGRALKDTTKMKPCYGSFAKPTNKEGG
QANLKDSEPSLSTEWSPCSVTCGNGIQVRTIVANYDPDIVMYTENVDLQT
Ad7 NANP HVR1

(SEQ ID NO: 3)

FDIRGVLDRGPSFKPYSGTAYNSLAPKGAPNTSQWIVTNANPNANPNANP
NANPSTKGDNYTFGIASTKGDNITKEGLEIGKDITADNKPIYADKTYQPE
PQVGEESWTDIDGTNEKFGGRALKPATKMKPCYGSFARPTNIKGGQAKNR
KVTPTEGDVEAEEPDIDMEFFDGREAADAFSPEIVLYTENVNLETPDSHV
VYKP
Ad7 NVDP HVR1

(SEQ ID NO: 4)

FDIRGVLDRGPSFKPYSGTAYNSLAPKGAPNTSQWIVTNANPNVDPNANP
NANPNANPSTKGDNYTFGIASTKGDNITKEGLEIGKDITADNKPIYADKT
YQPEPQVGEESWTDIDGTNEKFGGRALKPATKMKPCYGSFARPTNIKGGQ
AKNRKVTPTEGDVEAEEPDIDMEFFDGREAADAFSPEIVLYTENVNLETP
Ad7 NANP HVR2

(SEQ ID NO: 5)

FDIRGVLDRGPSFKPYSGTAYNSLAPKGAPNTSQWIVTTGESTKGDNYTF
GIASTKGDNANPNANPNANPNANPADNKPIYADKTYQPEPQVGEES WTD
IDGTNEKFGGRALKPATKMKPCYGSFARPTNIKGGQAKNRKVTPTEGDVE
AEEPDIDMEFFDGREAADAFSPEIVLYTENVNLETPDSHVVYKPGTSDGN
SHANL
Ad7 NVDP HVR2

(SEQ ID NO: 6)

FDIRGVLDRGPSFKPYSGTAYNSLAPKGAPNTSQWIVTTGESTKGDNYTF
GIASTKGDNANPNVDPNANPNANPNANPADNKPIYADKTYQPEPQVGEES
WTDIDGTNEKFGGRALKPATKMKPCYGSFARP‘I’NIKGGQAKNRKVTPT
EGDVEAEEPDIDMEFFDGREAADAFSPEIVLYTENVNLETPDSHVVYKPG
TS
Ad7 NANP HVR1; T* HVR5

(SEQ ID NO: 7)

FDIRGVLDRGPSFKPYSGTAYNSLAPKGAPNTSQWIVTNANPNANPNANP
NANPSTKGDNYTFGIASTKGDNITKEGLEIGKDITADNKPIYADKTYQPE
PQVGEESWTDIDGTNEKFGGRALKPATKMKPCYGSFARPTNIKGGQAKNR
KVTPSLSTEWSPCSVTCGNGIQVRREAADAFSPEIVLYTENVNLETPDSH
VVYK
Ad7 NVDP HVRI, T* HVR5

(SEQ ID NO: 8)

FDIRGVLDRGPSFKPYSGTAYNSLAPKGAPNTSQWIVTNANPNVDPNANP
NANPNANPSTKGDNYTFGIASTKGDNITKEGLEIGKDITADNKPIYADKT
YQPEPQVGEESWTDIDGTNEKFGGRALKPATKMKPCYGSFARPTNIKGGQ
AKNRKVTPSLSTEWSPCSVTCGNGIQVRREAADAFSPEIVLYTENVNLET
Ad5 NANP HVRI:

(SEQ ID NO: 9)

FDIRGVLDRGPTFKPYSGTAYNALAPKGAPNPCEWDEANANPNANPNANP
NANPVFGQAPYSGINITKEGIQIGVEGQTPKYADKTFQPEPQIGESQWYE
TEINHAAGRVLKKTTPMKPCYGSYAKPTNENGGQGILVKQQNGKLESQVE
MQFFSTTEATAGNGDNLTPKVVLYSEDVDIETPDTHISYMPTIKEGNSRE
LMGQ
Ad5 NVDP HVRI:

(SEQ ID NO: 10)

FDIRGVLDRGPTFKPYSGTAYNALAPKGAPNPCEWDEANANPNVDPNANP
NANPNANPVFGQAPYSGINITKEGIQIGVEGQTPKYADKTFQPEPQIGES
QWYETEINHAAGRVLKKTTPMKPCYGSYAKPTNENGGQGILVKQQNGKLE
SQVEMQFFSTTEATAGNGDNLTPKVVLYSEDVDIETPDTHISYMPTIKEG
Ad5 NANP HVR2

(SEQ ID NO: 11)

FDIRGVLDRGPTFKPYSGTAYNALAPKGAPNPCEWDEAATALEINLEEED
DDNEDEVDEQAEQQKTHVFGQAPYSGINITKEGNANPNANPNANPNANPT
FQPEPQIGESQWYETEINHAAGRVLKKTTPMKPCYGSYAKPTNENGGQGI
LVKQQNGKLESQVEMQFFSTTEATAGNGDNLTPKVVLYSEDVDIETPDTH
ISYM
Ad5 NVDP HVR2

(SEQ ID NO: 12)

FDIRGVLDRGPTFKPYSGTAYNALAPKGAPNPCEWDEAATALEINLEEED
DDNEDEVDEQAEQQKTHVFGQAPYSGINITKEGNANPNVDPNANPNANPN
ANPTFQPEPQIGESQWYETEINHAAGRVLKKTTPMKPCYGSYAKPTNENG
GQGILVKQQNGKLESQVEMQFFSTTEATAGNGDNLTPKVVLYSEDVDIET
Ad5 NANP HVRI; T* HVR5

(SEQ ID NO: 13)

FDIRGVLDRGPTFKPYSGTAYNALAPKGAPNPCEWDEANANPNANPNANP
NANPVFGQAPYSGINITKEGIQIGVEGQTPKYADKTFQPEPQIGESQWYE
TEINHAAGRVLKKTTPMKPCYGSYAKPTNENGGQGILVKQQNGKLESQVE
MQFFSTTESLSTEWSPCSVTCGNGIQVRTPKVVLYSEDVDIETPDTHISY
MPTIY
Ad5 NVDP HVR1, T* HVR5

(SEQ ID NO: 14)

FDIRGVLDRGPTFKPYSGTAYNALAPKGAPNPCEWDEANANPNVDPNANP
NANPNANPVFGQAPYSGINITKEGIQIGVEGQTPKYADKTFQPEPQIGES
QWYETEINHAAGRVLKKTTPMKPCYGSYAKPTNENGGQGILVKQQNGKLE
SQVEMQFFSTTESLSTEWSPCSVTCGNGIQVRTPKVVLYSEDVDIETPDT
Ad4 NANP HVR1

(SEQ ID NO: 15)

FDIRGVLDRGPSFKPYSGTAYNSLAPKGAPNTCQWKNANPNANPNANPSD
SKMHTFGAAAMPGVTGKKIEADGLPIRIDSTSGTDTVIYADKTFQPEPQV
GNDSWVDTNGAEEKYGGRALKDTTKMKPCYGSFAKPTNKEGGQANLKDSE
PAATTPNYDIDLAFFDSKTIVANYDPDIVMYTENVDLQTPDTHIVYKPGT
Ad4 NDVP HVR1

(SEQ ID NO: 16)

FDIRGVLDRGPSFKPYSGTAYNSLAPKGAPNTCQWKNANPNVDPNANPNA
NPNANPSDSKMHTFGAAAMPGVTGKKIEADGLPIRIDSTSGTDTVIYADK
TFQPEPQVGNDSWVDTNGAEEKYGGRALKDTTKMKPCYGSFAKPTNKEGG
QANLKDSEPAATTPNYDIDLAFFDSKTIVANYDPDIVMYTENVDLQTPDT
Ad4 NANP HVR2

(SEQ ID NO: 17)

FDIRGVLDRGPSFKPYSGTAYNSLAPKGAPNTCQWKDSDSKMHTFGAAAM
PGVNANPNANPNANPTDTVIYADKTFQPEPQVGNDSWVDTNGAEEKYGGR
ALKDTTKMKPCYGSFAKPTNKEGGQANLKDSEPAATTPNYDIDLAFFDSK
TIVANYDPDIVMYTENVDLQTPDTHIVYKPGTEDTSSESNLGQQAMPNRP
Ad4 NVDP HVR2

(SEQ ID NO: 18)

FDIRGVLDRGPSFKPYSGTAYNSLAPKGAPNTCQWKDSDSKMHTFGAAAM
PGVNANPNVDPNANPNANPNANPTDTVIYADKTFQPEPQVGNDSWVDTNG
AEEKYGGRALKDTTKMKPCYGSFAKPTNKEGGQANLKDSEPAATTPNYDI
DLAFFDSKTIVANYDPDIVMYTENVDLQTPDTHIVYKPGTEDTSSESNLG
Ad4 NANP HVR1; T* HVR5

(SEQ ID NO: 19)

FDIRGVLDRGPSFKPYSGTAYNSLAPKGAPNTCQWKNANPNANPNANPNA
NPSDSKMHTFGAAAMPGVTGKKIEADGLPIRIDSTSGTDTVIYADKTFQP
EPQVGNDSWVDTNGAEEKYGGRALKDTTKMKPCYGSFAKPTNKEGGQANL
KDSEPSLSTEWSPCSVTCGNGIQVRTIVANYDPDIVMYTENVDLQTPDTH
IVYK

In certain preferred embodiments, the CSP peptide segment selected from the group consisting of:

i) (NANP)ₙ where n is an integer from 3 to about 10 (SEQ ID NO:51);

ii) NANPNVDP(NANP)ₙ where n is an integer from 3 to about 8 (SEQ ID NO:52);

iii) related or consensus sequences derived from the P. falciparum CSP central repeat region (amino acids ˜105-272) up to about 40 amino acid residues in length;

iv) EYLNKIQNSLSTEWSPCSVT (T* epitope) (SEQ ID NO:53);

v) (GDRAAGQPA)ₙ where n is an integer from 2 to about 5 (SEQ ID NO:54);

vi) (ANGAGNQPG)ₙ where n is an integer from 2 to about 5 (SEQ ID NO:55);

vii) (APGANQEGGAA)ₙ where n is an integer from 2 to about 4 (SEQ ID NO:56); and

viii) related or consensus sequences derived from the P. vivax CSP central repeat region (amino acids ˜71-283) up to about 40 amino acid residues in length.

CSP sequences for P. vivax and P. falciparum can be found, e.g., in Arnot et al., Gonzalez et al., GenPept XP 001351122 and Hall et al.

Effective dosages for the pharmaceutical compositions and vaccines described herein can be determined by those of skill in the art without undue experimentation, and are expected to be in the range of 10⁴ to 10⁷ plaque-forming units per dose.

All publications, patents and patent applications disclosed herein are incorporated into this application by reference in their entirety.

This application claims priority to U.S. provisional application No. 60/854,876, filed Oct. 26, 2006, which is hereby incorporated by reference.

### EXAMPLES

### Construction of Capsid Display Recombinants

### Table 1

TABLE 1
Capsid display recombinants.

| | Insert/length | | |
|---|---|---|---|
| Name | (Amino acids) | HVR | Mode |
| G2 | PfCSP NANP/20 | HVR1 | substitution |
| G16 | PfCSP NANP/20 | HVR5 | insertion |
| I-i | PfCSP NVDP/24 | HVR1 | substitution |
| II-e | PfCSP T*/20 | HVR5 | substitution |
| II-g | PfCSP NVDP/24 | HVR1 | insertion |
| 1.5.18 | HPV16 L2/30 | HVR1 | substitution |
| 2.6.1 | HPV16 L2/30 | HVR5 | insertion |
| 2.7.6 | HPV16 L2/30 | HVR5 | substitution |

Abbreviations:
Pf: Plasmodum falciparum.
NANP: (NANP)₅ (SEQ ID NO: 57)
NVDP: NANPNVDP(NANP)₄ (SEQ ID NO: 58)
T*: EYLNKIQNSLSTEWSPCSVTI (SEQ ID NO: 53)
L2: HPV16 L2 amino acids 12-41;
RASATQLYKTCKQAG TCPPDIIPKVEGKTI (SEQ ID NO: 59).
Amino acids are indicated by the single-letter notation.

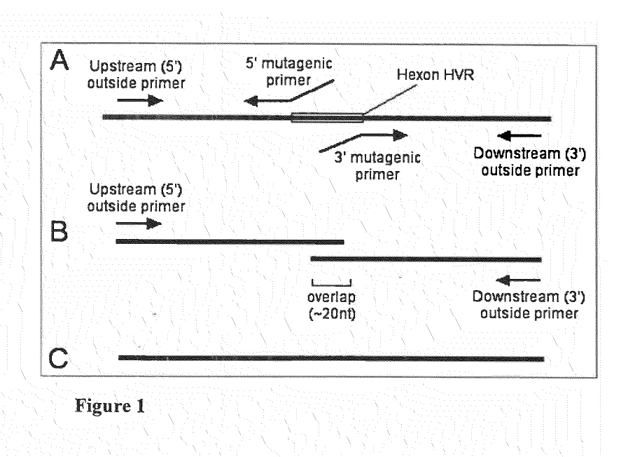

Hexon genes containing insertions and substitutions in hypervariable regions were constructed by overlap PCR (see, e.g. FIG. 1). For each modification, two separate first-round PCR reactions were performed, each using an ‘outside’ primer, either upstream (5′) or downstream (3′) of the portion of the hexon gene containing the targeted hypervariable region, and a mutagenic primer bearing a portion of the sequences to be inserted/substituted and hexon sequences immediately adjacent to the desired site of modification (FIG. 1A). The mutagenic primer sequences are chosen such that the products of the two first-round PCR reactions are DNA segments that overlap by about 20 nucleotides in the inserted/substituted region (FIG. 1B). The template for PCR was adenovirus virion DNA or a cloned segment of adenovirus DNA that includes the hexon gene.

A mixture of first-round PCR products was than used as template for a second round of PCR amplification employing the original outside primers. The result is a PCR product that spans the region from one outside primer to the other and contains the desired insertion or substitution mutation (FIG. 1C). Second round PCR fragments (about 1.5 kb in length) were cloned in the pCR2.1 vector (Invitrogen) and their nucleotide sequences were confirmed by DNA sequencing. The primers used in construction of the HPV L2 and P. falciparum CSP capsid display recombinants are given in Table 2, and hexon protein sequences in Table 3.

TABLE 2
Primer sequences used in constructing HPV L2 and P. falciparum CSP capsid display recombinants

| | | Sequence, 5′ to 3′. hexon sequences in CAPS; |
|---|---|---|
| Recombinant | Primer | insert/substitution sequences in lower case. |
| All | Upstream | CGGCGTGCTGGACAGGGGCCC (SEQ ID NO: 20) |
| | outside | |
| | Downstream | GCTGGCTCCGTCAACCC (SEQ ID NO: 21) |
| | outside | |
| G2 | 5′ mutagenic | cattcgggttagcgttaggatttgcgttgggattggcattAGCTTCATCCCATTCGCAAGGATTTGGGG |
| | | (SEQ ID NO: 22) |
| | 3′ mutagenic | tcctaacgctaacccgaatgcaaaccccaacgccaatcctGTATTTGGGCAGGCGCCTTATTCTGG |
| | | (SEQ ID NO: 23) |
| G16 | 5′ mutagenic | cattcgggttagcgttaggatttgcgttgggattggcattCTCAGTAGTTGAGAAAAATTGCATTTCCAC (SEQ ID |
| | | NO: 24) |
| | 3′ mutagenic | tcctaacgctaacccgaatgcaaaccccaacgccaatcctGCGACCGCAGGCAATGGTG (SEQ ID NO: 25) |
| I-i | 5′ mutagenic | gcattcgggttagcgttaggatttgcgttaggatcgacgttgggattggcattAGCTTCATCCCATTCGCAAGG |
| | | (SEQ ID NO: 26) |
| | 3′ mutagenic | tcctaacgctaacccgaatgcaaaccccaacgccaatcctGTATTTGGGCAGGCGCCTTATTCTGG |
| | | (SEQ ID NO: 27) |
| II-e | 5′ mutagenic | ccattcagtgctcagggaattctgaattttattcagatattccGCCTCAGTAGTTGAGAAAAATTGC |
| | | (SEQ ID NO: 28) |
| | 3′ mutagenic | gaattccctgagcactgaatggtcaccttgtagcgtgactTTGACTCCTAAAGTGGTATTG (SEQ ID NO: 29) |
| II-g | 5′ mutagenic | gcattcgggttagcgttaggatttgcgttaggatcgacgttgggattggcattAGCTTCATCCCATTCGCAAGG |
| | | (SEQ ID NO: 30) |
| | 3′ mutagenic | tcctaacgctaacccgaatgcaaaccccaacgccaatcctGCTACTGCTCTTGAAATAAACC (SEQ ID NO: 31) |
| 1.5.18 | 5′ mutagenic | cgggtgggcaggtgccggcctgcttgcaggtcttgtacagctgggtggcgctggctctAGCTTCATCCCATTCGCAAG |
| | | G (SEQ ID NO: 32) |
| | 3′ mutagenic | gcaggccggcacctgcccacccgatatcatccccaaggtggagggcaagaccatcGTATTTGGGCAGGCGCCTTATTC |
| | | TGG (SEQ ID NO: 33) |
| 2.6.1 | 5′ mutagenic | cgggtgggcaggtgccggcctgcttgcaggtcttgtacagctgggtggcgctggctctCTCAGTAGTTGAGAAAAATT |
| | | GC (SEQ ID NO: 34) |
| | 3′ mutagenic | gcaggccggcacctgcccacccgatatcatccccaaggtggagggcaagaccatcGCGACCGCAGGCAATGGT |
| | | (SEQ ID NO: 35) |
| 2.7.6 | 5′ mutagenic | cgggtgggcaggtgccggcctgcttgcaggtcttgtacagctgggtggcgctggctctCTCAGTAGTTGAGAAAAATT |
| | | GC (SEQ ID NO: 36) |
| | 3′ mutagenic | gcaggccggcacctgcccacccgatatcatccccaaggtggagggcaagaccatcACTCCTAAAGTGGTATTGTAC |
| | | (SEQ ID NO: 37) |

TABLE 3

P. falciparum CSP and HPV16 L2 capsid display hexon sequences.
Amino acid sequences of hexon proteins of capsid display
recombinants. Native hexon sequences are in CAPS, the
inserted/substituted CSP or HPV16 L2 sequences in lower case.

| | |
|---|---|
| Recombinant | Hexon protein sequence |

| | |
|---|---|
| G2 | MATPSMMPQWSYMHISGQDASEYLSPGLVQFARATETYFSLNNKFRNP |
| | TVAPTHDVTTDRSQRLTLRFIPVDREDTAYSYKARFTLAVGDNRVLDM |
| | ASTYFDIRGVLDRGPTFKPYSGTAYNALAPKGAPNPCEWDEAnanpnanp |
| | nanpnanpnanpGQAPYSGINITKEGIQIGVEGQTPKYADKTFQPEPQIGESQ |
| | WYETEINHAAGRVLKKTTPMKPCYGSYAKPTNENGGQGILVKQQNGK |
| | LESQVEMQFFSTTEATAGNGDNLTPKVVLYSEDVDIETPDTHISYMPTI |
| | KEGNSRELMGQQSMPNRPNYIAFRDNFIGLMYYNSTGNMGVLAGQAS |
| | QLNAVVDLQDRNTELSYQLLLDSIGDRTRYFSMWNQAVDSYDPDVRII |
| | ENHGTEDELPNYCFPLGGVINTETLTKVKPKTGQENGWEKDATEFSDK |
| | NEIRVGNNFAMEINLNANLWRNFLYSNIALYLPDKLKYSPSNVKISDNP |
| | NTYDYMNKRVVAPGLVDCYINLGARWSLDYMDNVNPFNHHRNAGLR |
| | YRSMLLGNGRYVPFHIQVPQKFFAIKNLLLLPGSYTYEWNFRKDVNMV |
| | LQSSLGNDLRVDGASIKFDSICLYATFFPMAHNTASTLEAMLRNDTNDQ |
| | SFNDYLSAANMLYPIPANATNVPISIPSRNWAAFRGWAFTRLKTKETPS |
| | LGSGYDPYYTYSGSIPYLDGTFYLNHTFKKVAITFDSSVSWPGNDRLLT |
| | PNEFEIKRSVDGEGYNVAQCNMTKDWFLVQMLANYNIGYQGFYIPESY |
| | KDRMYSFFRNFQPMSRQVVDDTKYKDYQQVGILHQHNNSGFVGYLAP |
| | TMREGQAYPANFPYPLIGKTAVDSITQKKFLCDRTLWRIPFSSNFMSMG |
| | ALTDLGQNLLYANSAHALDMTFEVDPMDEPTLLYVLFEVFDVVRVHR |
| | PHRGVIETVYLRTPFSAGNATT (SEQ ID NO: 38) |
| G16 | MATPSMMPQWSYMHISGQDASEYLSPGLVQFARATETYFSLNNKFRNP |
| | TVAPTHDVTTDRSQRLTLRFIPVDREDTAYSYKARFTLAVGDNRVLDM |
| | ASTYFDIRGVLDRGPTFKPYSGTAYNALAPKGAPNPCEWDEAATALEIN |
| | LEEEDDDNEDEVDEQAEQQKTHVFGQAPYSGINITKEGIQIGVEGQTPK |
| | YADKTFQPEPQIGESQWYETEINHAAGRVLKKTTPMKPCYGSYAKPTN |
| | ENGGQGILVKQQNGKLESQVEMQFFSTTEnanpnanpnanpnanpnanpATAG |
| | NGDNLTPKVVLYSEDVDIETPDTHISYMPTIKEGNSRELMGQQSMPNRP |
| | NYIAFRDNFIGLMYYNSTGNMGVLAGQASQLNAVVDLQDRNTELSYQ |
| | LLLDSIGDRTRYFSMWNQAVDSYDPDVRIIENHGTEDELPNYCFPLGGV |
| | INTETLTKVKPKTGQENGWEKDATEFSDKNEIRVGNNFAMEINLNANL |
| | WRNFLYSNIALYLPDKLKYSPSNVKISDNPNTYDYMNKRVVAPGLVDC |
| | YINLGARWSLDYMDNVNPFNHHRNAGLRYRSMLLGNGRYVPFHIQVP |
| | QKFFAIKNLLLLPGSYTYEWNFRKDVNMVLQSSLGNDLRVDGASIKFD |
| | SICLYATFFPMAHNTASTLEAMLRNDTNDQSFNDYLSAANMLYPIPAN |
| | ATNVPISIPSRNWAAFRGWAFTRLKTKETPSLGSGYDPYYTYSGSIPYLD |
| | GTFYLNHTFKKVAITFDSSVSWPGNDRLLTPNEFEIKRSVDGEGYNVAQ |
| | CNMTKDWFLVQMLANYNIGYQGFYIPESYKDRMYSFFRNFQPMSRQV |
| | VDDTKYKDYQQVGILHQHNNSGFVGYLAPTMREGQAYPANFPYPLIG |
| | KTAVDSITQKKFLCDRTLWRIPFSSNFMSMGALTDLGQNLLYANSAHA |
| | LDMTFEVDPMDEPTLLYVLFEVFDVVRVHRPHRGVIETVYLRTPFSAG |
| | NATT (SEQ ID NO: 39) |
| I-i | MATPSMMPQWSYMHISGQDASEYLSPGLVQFARATETYFSLNNKFRNP |
| | TVAPTHDVTTDRSQRLTLRFIPVDREDTAYSYKARFTLAVGDNRVLDM |
| | ASTYFDIRGVLDRGPTFKPYSGTAYNALAPKGAPNPCEWDEAnanpnvdp |
| | nanpnanpnanpnanpVFGQAPYSGINITKEGIQIGVEGQTPKYADKTFQPEPQ |
| | IGESQWYETEINHAAGRVLKKTTPMKPCYGSYAKPTNENGGQGILVKQ |
| | QNGKLESQVEMQFFSTTEATAGNGDNLTPKVVLYSEDVDIETPDTHISY |
| | MPTIKEGNSRELMGQQSMPNRPNYIAFRDNFIGLMYYNSTGNMGVLAG |
| | QASQLNAVVDLQDRNTELSYQLLLDSIGDRTRYFSMWNQAVDSYDPD |
| | VRIIENHGTEDELPNYCFPLGGVINTETLTKVKPKTGQENGWEKDATEF |
| | SDKNEIRVGNNFAMEINLNANLWRNFLYSNIALYLPDKLKYSPSNVKIS |
| | DNPNTYDYMNKRVVAPGLVDCYINLGARWSLDYMDNVNPFNHHRNA |
| | GLRYRSMLLGNGRYVPFHIQVPQKFFAIKNLLLLPGSYTYEWNFRKDV |
| | NMVLQSSLGNDLRVDGASIKFDSICLYATFFPMAHNTASTLEAMLRND |
| | TNDQSFNDYLSAANMLYPIPANATNVPISIPSRNWAAFRGWAFTRLKTK |
| | ETPSLGSGYDPYYTYSGSIPYLDGTFYLNHTFKKVAITFDSSVSWPGND |
| | RLLTPNEFEIKRSVDGEGYNVAQCNMTKDWFLVQMLANYNIGYQGFYI |
| | PESYKDRMYSFFRNFQPMSRQVVDDTKYKDYQQVGILHQHNNSGFVG |
| | YLAPTMREGQAYPANFPYPLIGKTAVDSITQKKFLCDRTLWRIPFSSNF |
| | MSMGALTDLGQNLLYANSAHALDMTFEVDPMDEPTLLYVLFEVFDVV |
| | RVHRPHRGVIETVYLRTPFSAGNATT (SEQ ID NO: 40) |
| II-e | MATPSMMPQWSYMHISGQDASEYLSPGLVQFARATETYFSLNNKFRNP |
| | TVAPTHDVTTDRSQRLTLRFIPVDREDTAYSYKARFTLAVGDNRVLDM |
| | ASTYFDIRGVLDRGPTFKPYSGTAYNALAPKGAPNPCEWDEAATALEIN |
| | LEEEDDDNEDEVDEQAEQQKTHVFGQAPYSGINITKEGIQIGVEGQTPK |
| | YADKTFQPEPQIGESQWYETEINHAAGRVLKKTTPMKPCYGSYAKPTN |
| | ENGGQGILVKQQNGKLESQVEMQFFSTTEAeylnkiqnslstewspcsvtLTPKV |
| | VLYSEDVDIETPDTHISYMPTIKEGNSRELMGQQSMPNRPNYIAFRDNFI |
| | GLMYYNSTGNMGVLAGQASQLNAVVDLQDRNTELSYQLLLDSIGDRT |
| | RYFSMWNQAVDSYDPDVRIIENHGTEDELPNYCFPLGGVINTETLTKVK |
| | PKTGQENGWEKDATEFSDKNEIRVGNNFAMEINLNANLWRNFLYSNIA |
| | LYLPDKLKYSPSNVKISDNPNTYDYMNKRVVAPGLVDCYINLGARWSL |
| | DYMDNVNPFNHHRNAGLRYRSMLLGNGRYVPFHIQVPQKFFAIKNLLL |
| | LPGSYTYEWNFRKDVNMVLQSSLGNDLRVDGASIKFDSICLYATFFPM |
| | AHNTASTLEAMLRNDTNDQSFNDYLSAANMLYPIPANATNVPISIPSRN |
| | WAAFRGWAFTRLKTKETPSLGSGYDPYYTYSGSIPYLDGTFYLNHTFK |
| | KVAITFDSSVSWPGNDRLLTPNEFEIKRSVDGEGYNVAQCNMTKDWFL |
| | VQMLANYNIGYQGFYIPESYKDRMYSFFRNFQPMSRQVVDDTKYKDY |
| | QQVGILHQHNNSGFVGYLAPTMREGQAYPANFPYPLIGKTAVDSITQK |
| | KFLCDRTLWRIPFSSNFMSMGALTDLGQNLLYANSAHALDMTFEVDP |
| | MDEPTLLYVLFEVFDVVRVHRPHRGVIETVYLRTPFSAGNATT (SEQ ID |
| | NO: 41) |
| II-g | MATPSMMPQWSYMHISGQDASEYLSPGLVQFARATETYFSLNNKFRNP |
| | TVAPTHDVTTDRSQRLTLRFIPVDREDTAYSYKARFTLAVGDNRVLDM |
| | ASTYFDIRGVLDRGPTFKPYSGTAYNALAPKGAPNPCEWDEAnanpnvdp |
| | nanpnanpnanpnanpATALEINLEEEDDDNEDEVDEQAEQQKTHVFGQAPY |
| | SGINITKEGIQIGVEGQTPKYADKTFQPEPQIGESQWYETEINHAAGRVL |
| | KKTTPMKPCYGSYAKPTNENGGQGILVKQQNGKLESQVEMQFFSTTEA |
| | TAGNGDNLTPKVVLYSEDVDIETPDTHISYMPTIKEGNSRELMGQQSMP |
| | NRPNYIAFRDNFIGLMYYNSTGNMGVLAGQASQLNAVVDLQDRNTEL |
| | SYQLLLDSIGDRTRYFSMWNQAVDSYDPDVRIIENHGTEDELPNYCFPL |
| | GGVINTETLTKVKPKTGQENGWEKDATEFSDKNEIRVGNNFAMEINLN |
| | ANLWRNFLYSNIALYLPDKLKYSPSNVKISDNPNTYDYMNKRVVAPGL |
| | VDCYINLGARWSLDYMDNVNPFNHHRNAGLRYRSMLLGNGRYVPFHI |
| | QVPQKFFAIKNLLLLPGSYTYEWNFRKDVNMVLQSSLGNDLRVDGASI |
| | KFDSICLYATFFPMAHNTASTLEAMLRNDTNDQSFNDYLSAANMLYPIP |
| | ANATNVPISIPSRNWAAFRGWAFTRLKTKETPSLGSGYDPYYTYSGSIP |
| | YLDGTFYLNHTFKKVAITFDSSVSWPGNDRLLTPNEFEIKRSVDGEGYN |
| | VAQCNMTKDWFLVQMLANYNIGYQGFYIPESYKDRMYSFFRNFQPMS |
| | RQVVDDTKYKDYQQVGILHQHNNSGFVGYLAPTMREGQAYPANFPYP |
| | LIGKTAVDSITQKKFLCDRTLWRIPFSSNFMSMGALTDLGQNLLYANSA |
| | HALDMTFEVDPMDEPTLLYVLFEVFDVVRVHRPHRGVIETVYLRTPFS |
| | AGNATT (SEQ ID NO: 42) |
| 1.5.18 | MATPSMMPQWSYMHISGQDASEYLSPGLVQFARATETYFSLNNKFRNP |
| | TVAPTHDVTTDRSQRLTLRFIPVDREDTAYSYKARFTLAVGDNRVLDM |
| | ASTYFDIRGVLDRGPTFKPYSGTAYNALAPKGAPNPCEWDEArasatqlykt |
| | ckqagtcppdiipkvegktiVFGQAPYSGINITKEGIQIGVEGQTPKYADKTFQPEP |
| | QIGESQWYETEINHAAGRVLKKTTPMKPCYGSYAKPTNENGGQGILVK |
| | QQNGKLESQVEMQFFSTTEATAGNGDNLTPKVVLYSEDVDIETPDTHIS |
| | YMPTIKEGNSRELMGQQSMPNRPNYIAFRDNFIGLMYYNSTGNMGVLA |
| | GQASQLNAVVDLQDRNTELSYQLLLDSIGDRTRYFSMWNQAVDSYDP |
| | DVRIIENHGTEDELPNYCFPLGGVINTETLTKVKPKTGQENGWEKDATE |
| | FSDKNEIRVGNNFAMEINLNANLWRNFLYSNIALYLPDKLKYSPSNVKI |
| | SDNPNTYDYMNKRVVAPGLVDCYINLGARWSLDYMDNVNPFNHHRN |
| | AGLRYRSMLLGNGRYVPFHIQVPQKFFAIKNLLLLPGSYTYEWNFRKD |
| | VNMVLQSSLGNDLRVDGASIKFDSICLYATFFPMAHNTASTLEAMLRN |
| | DTNDQSFNDYLSAANMLYPIPANATNVPISIPSRNWAAFRGWAFTRLKT |
| | KETPSLGSGYDPYYTYSGSIPYLDGTFYLNHTFKKVAITFDSSVSWPGN |
| | DRLLTPNEFEIKRSVDGEGYNVAQCNMTKDWFLVQMLANYNIGYQGF |
| | YIPESYKDRMYSFFRNFQPMSRQVVDDTKYKDYQQVGILHQHNNSGFV |
| | GYLAPTMREGQAYPANFPYPLIGKTAVDSITQKKFLCDRTLWRIPFSSN |
| | FMSMGALTDLGQNLLYANSAHALDMTFEVDPMDEPTLLYVLFEVFDV |
| | VRVHRPHRGVIETVYLRTPFSAGN |
| | ATT (SEQ ID NO: 43) |
| 2.6.1 | MATPSMMPQWSYMHISGQDASEYLSPGLVQFARATETYFSLNNKFRNP |
| | TVAPTHDVTTDRSQRLTLRFIPVDREDTAYSYKARFTLAVGDNRVLDM |
| | ASTYFDIRGVLDRGPTFKPYSGTAYNALAPKGAPNPCEWDEAATALEIN |
| | LEEEDDDNEDEVDEQAEQQKTHVFGQAPYSGINITKEGIQIGVEGQTPK |
| | YADKTFQPEPQIGESQWYETEINHAAGRVLKKTTPMKPCYGSYAKPTN |
| | ENGGQGILVKQQNGKLESQVEMQFFSTTErasatqlyktckqagtcppdiipkvegkti |
| | ATAGNGDNLTPKVVLYSEDVDIETPDTHISYMPTIKEGNSRELMGQQS |
| | MPNRPNYIAFRDNFIGLMYYNSTGNMGVLAGQASQLNAVVDLQDRNT |
| | ELSYQLLLDSIGDRTRYFSMWNQAVDSYDPDVRIIENHGTEDELPNYCF |
| | PLGGVINTETLTKVKPKTGQENGWEKDATEFSDKNEIRVGNNFAMEIN |
| | LNANLWRNFLYSNIALYLPDKLKYSPSNVKISDNPNTYDYMNKRVVAP |
| | GLVDCYINLGARWSLDYMDNVNPFNHHRNAGLRYRSMLLGNGRYVP |
| | FHIQVPQKFFAIKNLLLLPGSYTYEWNFRKDVNMVLQSSLGNDLRVDG |
| | ASIKFDSICLYATFFPMAHNTASTLEAMLRNDTNDQSFNDYLSAANML |
| | YPIPANATNVPISIPSRNWAAFRGWAFTRLKTKETPSLGSGYDPYYTYS |
| | GSIPYLDGTFYLNHTFKKVAITFDSSVSWPGNDRLLTPNEFEIKRSVDGE |
| | GYNVAQCNMTKDWFLVQMLANYNIGYQGFYIPESYKDRMYSFFRNFQ |
| | PMSRQVVDDTKYKDYQQVGILHQHNNSGFVGYLAPTMREGQAYPANF |
| | PYPLIGKTAVDSITQKKFLCDRTLWRIPFSSNFMSMGALTDLGQNLLYA |
| | NSAHALDMTFEVDPMDEPTLLYVLFEVFDVVRVHRPHRGVIETVYLRT |
| | PFSAGNATT (SEQ ID NO: 44) |
| 2.7.6 | MATPSMMPQWSYMHISGQDASEYLSPGLVQFARATETYFSLNNKFRNP |
| | TVAPTHDVTTDRSQRLTLRFIPVDREDTAYSYKARFTLAVGDNRVLDM |
| | ASTYFDIRGVLDRGPTFKPYSGTAYNALAPKGAPNPCEWDEAATALEIN |
| | LEEEDDDNEDEVDEQAEQQKTHVFGQAPYSGINITKEGIQIGVEGQTPK |
| | YADKTFQPEPQIGESQWYETEINHAAGRVLKKTTPMKPCYGSYAKPTN |
| | ENGGQGILVKQQNGKLESQVEMQFFSTTErasatqlyktckqagtcppdiipkvegkti |
| | TPKVVLYSEDVDIETPDTHISYMPTIKEGNSRELMGQQSMPNRPNYIAF |
| | RDNFIGLMYYNSTGNMGVLAGQASQLNAVVDLQDRNTELSYQLLLDSI |
| | GDRTRYFSMWNQAVDSYDPDVRIIENHGTEDELPNYCFPLGGVINTETL |
| | TKVKPKTGQENGWEKDATEFSDKNEIRVGNNFAMEINLNANLWRNFL |
| | YSNIALYLPDKLKYSPSNVKISDNPNTYDYMNKRVVAPGLVDCYINLG |
| | ARWSLDYMDNVNPFNHHRNAGLRYRSMLLGNGRYVPFHIQVPQKFFA |
| | IKNLLLLPGSYTYEWNFRKDVNMVLQSSLGNDLRVDGASIKFDSICLYA |
| | TFFPMAHNTASTLEAMLRNDTNDQSFNDYLSAANMLYPIPANATNVPI |
| | SIPSRNWAAFRGWAFTRLKTKETPSLGSGYDPYYTYSGSIPYLDGTFYL |
| | NHTFKKVAITFDSSVSWPGNDRLLTPNEFEIKRSVDGEGYNVAQCNMT |
| | KDWFLVQMLANYNIGYQGFYIPESYKDRMYSFFRNFQPMSRQVVDDT |
| | KYKDYQQVGILHQHNNSGFVGYLAPTMREGQAYPANFPYPLIGKTAV |
| | DSITQKKFLCDRTLWRIPFSSNFMSMGALTDLGQNLLYANSAHALDMT |
| | FEVDPMDEPTLLYVLFEVFDVVRVHRPHRGVIETVYLRTPFSAGNATT |
| | (SEQ ID NO: 45) |

Modified hexon DNA segments were either subcloned into a plasmid carrying a larger segment of viral DNA or excised from pCR2.1 for use directly in recombination to produce intact viral genomes.

Hexon DNA segments containing insertions/substitutions were introduced into intact viral genomes by recombination between modified hexon DNA and adenovirus genomic DNA either in cells in tissue culture or in bacteria. For recombination in tissue culture, the hexon fragment and adenovirus genomic DNA singly cleaved at an Nde I site within the hexon gene were introduced into a standard adenovirus host cell line (293) by Ca₂PO₄ transfection. Recombination between the restriction fragment and the viral DNA generated viable, full-length viral genomes that propagated in the transfected culture and were recovered by plaque purification. For recombination in bacterial cells, the hexon fragment and a full-length adenovirus genomic plasmid, also cleaved once in the hexon gene, were electroporated into recombination-proficient E. coli, where recombination generated a circular plasmid that conferred antibiotic resistance. Virus was then recovered by transfection of 293 cells with purified plasmid DNA cleaved with Pac I to release the viral genome from the vector sequences. Both techniques yield both wild type and hexon-modified viral genomes, and either plaques (in tissue culture experiments) or plasmid preparations (in bacteria) must be examined to identify recombinants with the desired hexon structure. Therefore, a novel restriction site is incorporated into each insertion or substitution to facilitate screening. The recipient genomic DNA in both cases was obtained from plasmid CP08. CP08 was derived from pTG3602 (Transgene, S.A.) by removal of the Nde I site in fiber by a silent mutation, and insertion of a segment of the lacZ gene at the remaining Nde I site in hexon.

### Characterization of Capsid Display Recombinants.

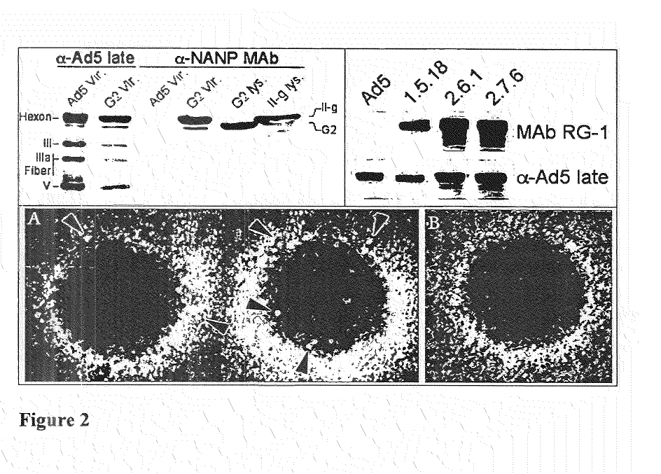

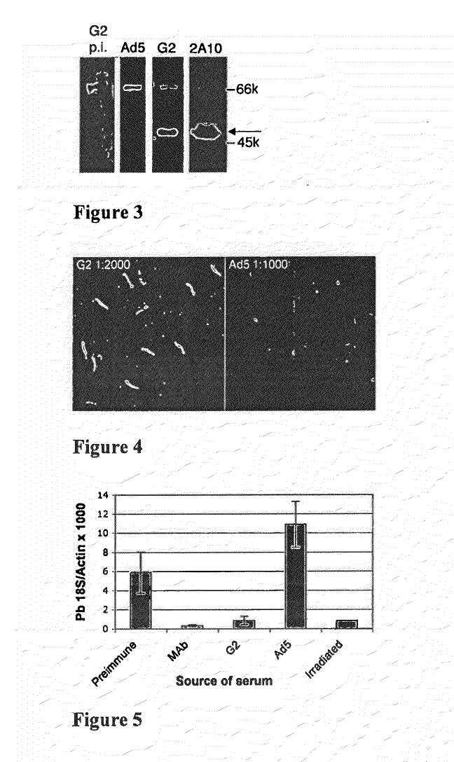

Monoclonal antibodies are available both to the P. falciparum CSP NANP repeat and to the peptide displayed by HPV L2 recombinants. Therefore, the hexon proteins of two NANP recombinants and all three HPV L2 recombinants were analyzed by immunoblotting to confirm the presence of the inserted peptide in hexon. All recombinants were reactive, as expected (FIG. 2). We also examined virions produced by the NANP recombinant G2 by immunoelectron microscopy, using the NANP monoclonal antibody and a gold-conjugated anti-mouse IgG secondary antibody. Recombinant virions are strongly gold-labeled (FIG. 3) but wild type Ad5 is not, indicating that the NANP epitope is exposed on the virion surface.

Malaria CSP capsid-display recombinants induce neutralizing antibody in mice. We expect capsid display recombinant virus particles to be immunogenic in mice despite their inability to replicate. To confirm that expectation we immunized mice with NANP recombinant G2. Mice were immunized intraperitoneally with three doses of 10¹⁰ CsCl gradient-purified particles at three-week intervals. Control mice each received 10¹⁰ particles of antigenically wild type Ad5 hr404 on the same schedule. Sera were obtained prior to immunization and two weeks after each injection. Additional sera were obtained at weeks 11 and 14 post-immunization.

Pooled sera from mice immunized with the G2 recombinant were first examined for anti-CSP antibody by ELISA, using a bacterially-produced recombinant P. falciparum CSP NANP-containing protein (MR4 MRA-272) as the capture antigen. The pooled G2 sera displayed a titer of 1:32,000 after the initial immunization and 1:64,000 after the second. The titer did not increase after the third injection. As expected, the Ad5-immunized mice produced no antibody reactive with recombinant CSP (titer <1:100 and indistinguishable from the pre-immunization serum). In an independent replicate experiment, ELISA titers of 1:64,000-1:128,000 were observed in individual mice after two injections. ELISA titers induced by G2 persisted for at least 14 weeks at a level indistinguishable from that at the five-week time point.

To confirm that the antibody induced by G2 reacts with authentic CSP, pooled sera were used in immunoblots to probe lysates of sporozoites dissected from the salivary glands of mosquitoes infected with a transgenic P. berghei strain that expresses a CSP protein containing the P. falciparum NANP region (Nardin et al., 1982) Pooled sera from G2-immunized mice and an anti-P. falciparum NANP monoclonal antibody (2A10, Nardin et al., 1982), but not pre-immune serum or serum from Ad5-immunized mice, recognize a sporozoite protein of the molecular weight predicted by the amino acid sequence of the chimeric protein (FIG. 3). Finally, the pooled sera from immunized mice were used in an indirect immunofluorescence experiment to stain previously frozen, intact P. falciparum sporozoites. The pooled G2 sera produced a detectable signal at a dilution of 1:8000 (1:2000 shown in FIG. 4), while MAb 2A10 was positive at 1:16,000. Ad5 serum produced no recognizable signal at 1:1000. These data confirm that recombinant adenovirus particles that display the CSP NANP repeat are capable of inducing high-titer antibody responses against both recombinant and native CSP.

One of the intents of immunization with CSP recombinants is to induce antibodies that neutralize sporozoites prior to the invasion of liver cells. Therefore, we investigated whether the antibodies induced by G2 were capable of neutralizing sporozoites in a quantitative in vitro sporozoite neutralizing assay (TSNA) (Kumar et al., 2004). In TSNA experiments, sera are incubated with live sporozoites, which are then permitted to invade liver cells in tissue culture. Parasite replication is measured by quantitative PCR (qPCR) determination of Plasmodium ribosomal 18S RNA (rRNA) sequences in total RNA extracted from the infected cells. In our experiments, pooled G2- or Ad5-immunized sera, pooled pre-immunization sera from G2-immunized mice, or 2A10 monoclonal antibody were incubated for 30 minutes at a 1:6 dilution with 20,000 sporozoites dissected from mosquitoes infected with the transgenic P. berghei/P. falciparum CSP strain. The mixture was added to HepG2 human liver cells and the sporozoites were allowed to invade and replicate. 72 h after infection, total RNA was extracted from the cells and P. berghei rRNA was measured by qPCR. Experiments were conducted with sera collected after two doses of recombinant virus in two independent courses of immunization. Data from one is presented in FIG. 5; essentially identical results were obtained in the second. In each experiment, serum from G2-immunized mice substantially (−6-fold) reduced sporozoite infectivity compared to preimmune serum pools. Neutralization by G2 was comparable to that by the NANP monoclonal antibody. We conclude from these experiments that NANP capsid display recombinants are highly immunogenic and that they induce antibodies that both recognize and neutralize sporozoites.

### HPV L2 CSP Capsid-Display Recombinants Induce Neutralizing Antibody and are Protective in Mice.

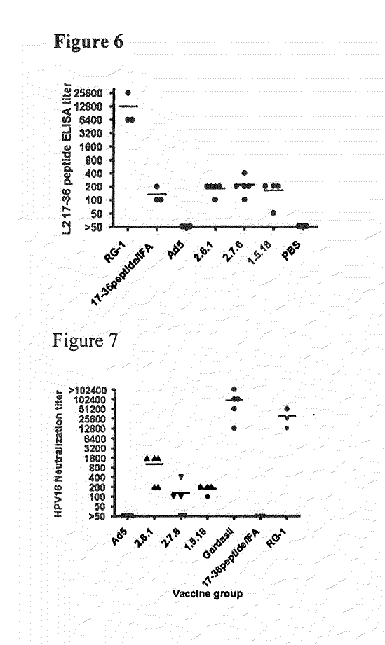

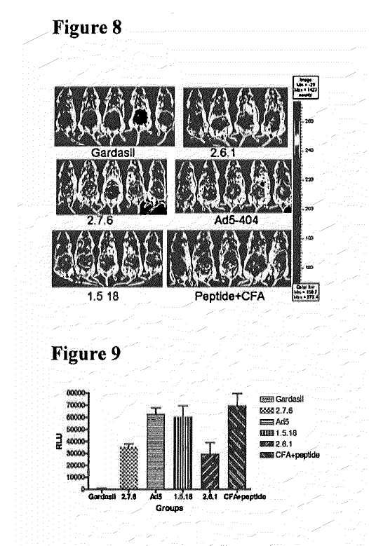

Three recombinants that express an epitope from the human papillomavirus 16 (HPV16) L2 protein were also examined for immunogenicity. Groups of 5 mice were each immunized i.p. as described above with 10¹⁰ recombinant adenovirus particles with no adjuvant, 20% of a vial of Gardasil, PBS, or 100 ug L2 17-36 peptide in complete Freund's adjuvant (CFA) for first immunization and incomplete Freund's adjuvant IFA for two boosts on days 14 and 28. Bleeds were taken on days 21 and 42, and the mice were challenged with HPV16 pseudovirions on day 44. The titer of HPV16 L2 17-36 peptide-specific serum antibodies was determined using the sera harvested on day 21 (FIG. 6). The positive control monoclonal antibody RG-1 bound to HPV16 L2 17-36 and serum antibody from mice vaccinated with PBS or adenovirus did not. However, low titers of serum antibodies were detected in all other vaccine groups suggesting that vaccination was successful. The mice received a second boost and sera harvested 14 days later were tested for their ability to neutralize HPV16 pseudovirions in vitro (FIG. 7). RG-1 tissue culture supernatant effectively neutralized the HPV16 pseudovirus validating the assay and demonstrating the presence of L2 in the pseudovirions. Sera from mice vaccinated with Gardasil (which contains HPV16 L1 VLPs) neutralized HPV16 pseudovirions at high titer, whereas mice vaccinated with adenovirus failed to detectably neutralize. Vaccination with HPV16 L2 17-36 peptide in CFA/IFA failed to induce neutralizing antibodies suggesting that it does not take up the appropriate conformation in solution or lacks sufficient T cell help to mount a neutralizing antibody response. However, sera from mice vaccinated with each of the recombinant adenoviruses neutralized HPV16, although at a titer lower than the sera obtained from mice vaccinated with Gardasil. We recently showed that L2-specific neutralizing antibody is sufficient to confer protection on naïve mice (Gambhira et al, 2007 in press). Therefore, immunized mice were challenged on their belly with HPV16 pseudovirions carrying the luciferase reporter at 16 days after the last immunization. Three days later cutaneous HPV16 pseudovirus infection was measured as bioluminescence in relative light units after injection of the mice with luciferin (FIGS. 8 and 9). Mice vaccinated with adenovirus were not protected, whereas those vaccinated with Gardasil were completely protected. Neither vaccination with the L2.17-36 peptide nor adenovirus provided statistically significant protection. However, there was evidence of significant protection against cutaneous HPV16 pseudovirus challenge of mice vaccinated with recombinant adenoviruses 2.6.1 (P<0.05, one way ANOVA, Tukey's post-test versus adenovirus, and P<0.01 versus L2 17-36 peptide) and 2.7.6 (P<0.05, one way ANOVA, Tukey's post-test versus L2 17-36 peptide). This suggests that the Adenovirus constructs display the neutralizing epitope more appropriately than peptide in CFA/IFA to induce a protective immunity.

Sambrook et al, Molecular Cloning. A Laboratory Manual (volumes I-III) 1989, Cold Spring Harbor Laboratory Press, USA” and “Harlow and Lane, Antibodies a Laboratory Manual 1988 and 1998, Cold Spring Harbor Laboratory Press, USA” provide sections describing methodology for antibody generation and purification, diagnostic platforms, cloning procedures, etc. that may be used in the practice of the instant invention.

### REFERENCES CITED HEREIN ARE LISTED BELOW FOR CONVENIENCE

Arnot D E, Barnwell J W, Tam J P, Nussenzweig V, Nussenzweig R S, Enea V. (1985) Circumsporozoite protein of Plasmodium vivax: gene cloning and characterization of the immunodominant epitope. Science, 230:815-8.
Berg, M., J. DiFatta, E. Hoiczyk, R. Schlegel, and G. Ketner. Viable adenovirus vaccine prototypes: High-level production of a papillomavirus capsid antigen from the major late transcriptional unit. Proc. Nat. Acad. Sci. (USA). 102:4590-4595 (2005).
Berg, M., R. Gambhira, M. Siracusa, E. Hoiczyk, R. Roden and G. Ketner. HPV16 L1 Capsid Protein Expressed from Viable Adenovirus Recombinants Elicits Neutralizing Antibody in Mice. Vaccine, 25:3501-3510 (2007).
Birkett, A., K. Lyons, et al. (2002). “A modified hepatitis B virus core particle containing multiple epitopes of the Plasmodium falciparum circumsporozoite protein provides a highly immunogenic malaria vaccine in preclinical analyses in rodent and primate hosts.” Infect Immun 70(12): 6860-70.
Bruna-Romero, O., G. Gonzalez-Aseguinolaza, et al. (2001). “Complete, long-lasting protection against malaria of mice primed and boosted with two distinct viral vectors expressing the same plasmodial antigen.” Proc Natl Acad Sci USA 98(20): 11491-6.
Crompton, J., C. 1. Toogood, et al. (1994). “Expression of a foreign epitope on the surface of the adenovirus hexon.” J Gen Virol 75 (Pt 1): 133-9.
Doherty, J. F., M. Pinder, et al. (1999). “A phase I safety and immunogenicity trial with the candidate malaria vaccine RTS,S/SBAS2 in semi-immune adults in The Gambia.” Am J Trop Med Hyg 61(6): 865-8.
Gambhira R, Jagu S, Karanam B, Gravitt P E, Culp T D, Christensen N D, Roden R B. Protection of Rabbits against Challenge with Rabbit Papillomaviruses by Immunization with the N Terminus of Human Papillomavirus Type 16 Minor Capsid Antigen L2. J Virol. 2007 November; 81(21):11585-92. Epub 2007 Aug. 22.
Gambhira R, Karanam B, Jagu S, Roberts J N, Buck C B, Bossis I, Alphs H, Culp T, Christensen N D, Roden R B. A protective and broadly cross-neutralizing epitope of Human Papillomavirus L2. J Virol. 2007 Oct. 10; [Epub ahead of print]
Gilbert, S. C., J. Schneider, et al. (2002). “Enhanced CD8 T cell immunogenicity and protective efficacy in a mouse malaria model using a recombinant adenoviral vaccine in heterologous prime-boost immunisation regimes.” Vaccine 20(7-8): 1039-45.
Gonzalez, J. M. et al. (2001) Variants of the Plasmodium vivax circumsporozoite protein (VK210 and VK247) in Colombian isolates. Mem. Inst. Oswaldo Cruz 96:709-712.
Hall, N., Pain, A., Berriman, M., Churcher, C., Harris, B., Harris, D., Mungall, K., Bowman, S., Atkin, R., Baker, S., Barron, A., Brooks, K., Buckee, C. O., Burrows, C., Cherevach, I., Chillingworth, C., Chillingworth, T., Christodoulou, Z., Clark, L., Clark, R., Corton, C., Cronin, A., Davies, R., Davis, P., Dear, P., Dearden, F., Doggett, J., Feltwell, T., Goble, A., Goodhead, I., Gwilliam, R., Hamlin, N., Hance, Z., Harper, D., Hauser, H., Hornsby, T., Holroyd, S., Horrocks, P., Humphray, S., Jagels, K., James, K. D., Johnson, D., Kerhornou, A., Knights, A., Konfortov, B., Kyes, S., Larke, N., Lawson, D., Lennard, N., Line, A., Maddison, M., Mclean, J., Mooney, P., Moule, S., Murphy, L., Oliver, K., Ormond, D., Price, C., Quail, M. A., Rabbinowitsch, E., Rajandream, M. A., Rutter, S., Rutherford, K. M., Sanders, M., Simmonds, M., Seeger, K., Sharp, S., Smith, R., Squares, R., Squares, S., Stevens, K., Taylor, K., Tivey, A., Unwin, L., Whitehead, S., Woodward, J., Sulston, J. E., Craig, A., Newbold, C. and Barrell, B. G. Sequence of Plasmodium falciparum chromosomes 1, 3-9 and 13. Nature 419 (6906), 527-531 (2002).
Kumar, K. A., et al., 2004, Quantitative Plasmodium sporozoite neutralization assay (TSNA). J Immunol Methods. 292: p. 157-64.
Moreno, A., P. Clavijo, et al. (1993). “CD4+ T cell clones obtained from Plasmodium falciparum sporozoite-immunized volunteers recognize polymorphic sequences of the circumsporozoite protein.” J Immunol 151(1): 489-99.
Nardin, E. H., et al., 1982, Circumsporozoite proteins of human malaria parasites Plasmodium falciparum and Plasmodium vivax. J Exp Med. 156: p. 20-30.
Nardin, E., F. Zavala, et al. (1999). “Pre-erythrocytic malaria vaccine: mechanisms of protective immunity and human vaccine trials.” Parassitologia 41(1-3): 397-402.
Nardin, E. H., J. M. Calvo-Calle, et al. (2001). “A totally synthetic polyoxime malaria vaccine containing Plasmodium falciparum B cell and universal T cell epitopes elicits immune responses in volunteers of diverse HLA types.” J Immunol 166(1): 481-9.
Nardin, E. H., D. A. Herrington, et al. (1989). “Conserved repetitive epitope recognized by CD4+ clones from a malaria-immunized volunteer.” Science 246(4937): 1603-6.
Nardin, E. H., G. A. Oliveira, et al. (2004). “Phase I testing of a malaria vaccine composed of hepatitis B virus core particles expressing Plasmodium falciparum circumsporozoite epitopes.” Infect Immun 72(11): 6519-27.
Persson, C., et al., 2002, Cutting edge: a new tool to evaluate human pre-erythrocytic malaria vaccines: rodent parasites bearing a hybrid Plasmodium falciparum circumsporozoite protein. J Immunol. 169: p. 6681-5.
Rodrigues, E. G., F. Zavala, et al. (1997). “Single immunizing dose of recombinant adenovirus efficiently induces CD8+ T cell-mediated protective immunity against malaria.” J Immunol 158(3): 1268-74.
Rodrigues, E. G., F. Zavala, et al. (1998). “Efficient induction of protective anti-malaria immunity by recombinant adenovirus.” Vaccine 16(19): 1812-7.
Rux, J. J., P. R. Kuser, et al. (2003). “Structural and phylogenetic analysis of adenovirus hexons by use of high-resolution x-ray crystallographic, molecular modeling, and sequence-based methods.” J Virol 77(17): 9553-66.
Walther, M., S. Dunachie, et al. (2005). “Safety, immunogenicity and efficacy of a pre-erythrocytic malaria candidate vaccine, ICC-1132 formulated in Seppic ISA 720.” Vaccine 23(7): 857-64.
Worgall, S, Krause, A et al. (2005) “Protection against P. aeruginoso with an adenovirus vector containing an OprF epitope in the capsid” J. Clinical Investigation Published online April 1 (http://www jci.org)
Zavala, F., J. P. Tam, et al. (1985). “Rationale for development of a synthetic vaccine against Plasmodium falciparum malaria.” Science 228(4706): 1436-40.

1. A recombinant adenovirus comprising a polynucleotide encoding a Papillomavirus L2 peptide segment, or a consensus sequence thereof, or a human malaria circumsporozoite protein (CSP) segment, or a consensus sequence thereof.

2. The recombinant adenovirus of claim 1 wherein said L2 or CSP polynucleotide is inserted into or replaces at least a portion of a DNA sequence encoding an adenovirus surface protein.

3. The recombinant adenovirus of claim 2 wherein said L2 or CSP polynucleotide is flanked by at least one spacer polynucleotide.

4-5. (canceled)

6. The recombinant adenovirus of claim 3 wherein said spacer polynucleotide is joined to the 3′ end and the 5′ end of said L2 or CSP polynucleotide.

7. The recombinant adenovirus of claim 6 wherein said spacer polynucleotide encodes a peptide tag.

8-34. (canceled)

35. A pharmaceutical composition comprising the recombinant adenovirus of claim 1.

36. The pharmaceutical composition of claim 35 that is a vaccine.

37. A method of vaccinating against human papillomavirus comprising administering an effective amount of the pharmaceutical composition of claim 35 to a subject.

38. (canceled)

39. The recombinant adenovirus of claim 1 comprising a CSP peptide segment selected from the group consisting of: i) (NANP)ₙ where n is an integer from 3 to 10 (SEQ ID NO:51); ii) NANPNVDP(NANP)ₙ where n is an integer from 3 to 8 (SEQ ID NO:52); iii) a peptide segment from the P. falciparum CSP central repeat region (amino acids −105-272); iv) EYLNKIQNSLSTEWSPCSVT (SEQ ID NO:53); v) (GDRAAGQPA)ₙ where n is an integer from 2 to 5 (SEQ ID NO:54); vi) (ANGAGNQPG)ₙ where n is an integer from 2 to 5 (SEQ ID NO:55); vii) (APGANQEGGAA)ₙ where n is an integer from 2 to 4 (SEQ ID NO:56); viii) a peptide segment from the P. vivax CSP central repeat region (amino acids −71-283).

40. The recombinant adenovirus of claim 39 wherein said CSP peptide segment is inserted into or replaces a portion of an adenoviral surface protein selected from the group consisting of: a) hexon; b) fiber; and c) protein IX capsid proteins.

41-42. (canceled)

43. The recombinant adenovirus of claim 40 wherein the peptide segment is inserted into or replaces a portion of fiber HI loop.

44-45. (canceled)

46. The recombinant adenovirus of claim 43 wherein the peptide segment is inserted into or replaces at least a portion of human adenovirus type 2 fiber HI loop amino acids 537-550, human adenovirus type 4 fiber HI loop amino acids 385-393, human adenovirus type 5 fiber HI loop amino acids 537-549, human adenovirus type 7 fiber HT loop amino acids 278-287, human adenovirus type 21 fiber HT loop amino acids 277-286, human adenovirus type 35 fiber HI loop amino acids 277-286, chimpanzee adenovirus type AdC7 fiber HI loop amino acids 403-411, chimpanzee adenovirus type AdC68 fiber HI loop amino acids 385-393.

47. The recombinant adenovirus of claim 46 wherein said adenovirus is capable of replicating in human cells.

48. The recombinant adenovirus of claim 47 wherein said adenovirus is capable of replicating in a mammalian host.

49. The recombinant adenovirus of claim 48 wherein said mammalian host is a human.

50. The recombinant adenovirus of claim 49 wherein said adenovirus is not capable of replicating in human cells.

51. The recombinant adenovirus of claim 50 wherein said adenovirus is capable of inducing an immune response.

52-54. (canceled)

55. A pharmaceutical composition comprising the recombinant adenovirus of claim 39.

56. The pharmaceutical composition of claim 55 that is a vaccine.

57. A method of vaccinating against malaria comprising administering an effective amount of the pharmaceutical composition of claim 55 to a subject.

58-59. (canceled)
